(19) United States
(12) Patent Application Publication
(10) Pub. No.: US 2024/0038333 A1
(43) Pub. Date: Feb. 1, 2024

(54) NUCLEIC ACID SEQUENCE ANALYSIS AND CONFIGURABLE REPORT GENERATION

(71) Applicants: ILLUMINA SOFTWARE, INC., San Diego, CA (US); ILLUMINA, INC., San Diego, CA (US)

(72) Inventors: Sam Ng, Foster City, CA (US); Dylan Barfield, New York, NY (US); Jing Gao, San Diego, CA (US); Kevin P. Rhodes, San Diego, CA (US); Sachin Parikh, San Diego, CA (US); Akshay Kotadia, San Diego, CA (US); Kim Pelak, San Diego, CA (US)

(21) Appl. No.: 18/357,829

(22) Filed: Jul. 24, 2023

Related U.S. Application Data
(60) Provisional application No. 63/444,827, filed on Feb. 10, 2023.
(60) Provisional application No. 63/369,478, filed on Jul. 26, 2022.

(51) Int. Cl. G16B 30/00; G16B 50/30

(52) U.S. Cl. CPC G16B 30/00; G16B 50/30

(57) ABSTRACT

The presently described techniques relate generally to configuration and use of a software platform that provides tools for users to store, arrange, and visualize genetic data, such as may be derived from a nucleic acid sequencing device. In addition, such a software platform may include one or more tools that allow a user to annotate genetic data with information available from external and/or internal genetic databases and to create custom reports based on such information. In practice, the software platform may be generic with respect to the sequencing device generating the sequence data, one or more upstream analytic packages, such as may perform variant identification or calling, and one or more external or internal data stores (e.g., knowledge bases or databases) used to access information about the sequence and/or variants identified therein.

### CROSS REFERENCE TO RELATED APPLICATIONS

This application claims the benefit of U.S. Provisional Application Ser. No. 63/444,827, entitled “NUCLEIC ACID SEQUENCE ANALYSIS AND CONFIGURABLE REPORT GENERATION,” filed Feb. 10, 2023, and U.S. Provisional Application Ser. No. 63/369,478, entitled “NUCLEIC ACID SEQUENCE ANALYSIS AND CONFIGURABLE REPORT GENERATION,” filed Jul. 26, 2022, which are hereby incorporated by reference in their entirety for all purposes.

### TECHNICAL FIELD

The present approach relates generally to the use of an application configured or coded to receive an output from a nucleic acid sequencing device (i.e., a sequencer) and to access internal or external data stores to facilitate downstream analysis of the output by one or more users. In certain aspects, the application supports configurable report generation by the user(s) so as to allow a customized report to be generated based on the sequencer output. In practice, the application may be generic as to the type(s) of sequence data that may be processed, the data stores (both internal and external) that may be accessed, and the prior data that may be relied upon in generating the customized report.

### BACKGROUND

In a nucleic acid sequencing context, a biological sample containing nucleic acid (e.g., DNA or RNA) may be input into and processed by a sequencing instrument capable of processing the sample and outputting a corresponding nucleic acid sequence. In practice, such as sequence may be analyzed to identify variants within the sample. The identified variants may be further analyzed to identify those that may be of interest for research, clinical, diagnostic, and/or therapeutic purposes.

In practice, such analyses may be complex and may involve a multitude of factors that may require assessment by one or more reviewers. In particular, the genome of an individual, and the number of variants needing assessment, may be extensive and the data sources used to evaluate identified variants may themselves be numerous, varied, and in some instances redundant. By way of example, numerous third-party or otherwise external data stores may be available to an individual assessing a DNA sequence for variants of interest that may be implicated in a genetic disease or disorder. However, such external data stores may utilize different database layouts and/or fields, may utilize different terminology or language cues, and/or may utilize different alert or action schemas, which may make assessment of a variant time-consuming. Further, the individual performing the review (or an organization they are affiliated with) may themselves have access to a history of prior work or cases that may be relevant to the review process. Such prior history may be informal notes or an organized and structured data store, that may itself differ in layout and terminology from external or third-party data stores relied upon by the individual. As a result, meaningful and straightforward analysis of the output of a nucleic acid sequencing device for useful insights may be an involved process to the extent that a variety of data stores, both internal and external, may need to be accessed and evaluated.

### SUMMARY

The present techniques provide for a software platform (e.g., a software application implemented locally (e.g., on-premise) or in a distributed (e.g., cloud implementation) manner and that provides tools for users to store, arrange, and visualize genetic data, such as may be derived from a nucleic acid sequencing device. In addition, such a software platform may include one or more tools that allow a user to annotate genetic data with information available from external and/or internal genetic databases and to create custom reports based on such information. In practice, the software platform may be generic with respect to the sequencing device generating the sequence data, one or more upstream analytic packages, such as may perform variant identification or calling, and one or more external or internal data stores (e.g., knowledge bases or databases) used to access information about the sequence and/or variants identified therein.

In one embodiment, one or more computer readable media are provided comprising machine-executable instructions. The machine-executable instructions, when executed, cause acts to be performed comprising: receiving as a first input a nucleic acid sequence dataset, wherein the nucleic acid sequence data set is an output of one or both of a primary analysis or a secondary analysis; displaying a selectable listing of one or more variants identified in the nucleic acid sequence dataset; receiving a selection of a variant of interest from the selectable listing of the one or more variants; accessing one or more data stores comprising variant data associated with the selection of the variant of interest; displaying one or more variant findings accessed from the one or more data stores; receiving a selection of one or more of the variant findings; creating an assertion for the variant of interest for each selection of the one or more variant findings; and generating a customized report based on the assertions.

In a further embodiment, one or more computer readable media are provided comprising machine-executable instructions. The machine-executable instructions, when executed, cause acts to be performed comprising: accessing or receiving a data file comprising genetic data for a subject, wherein the genetic data comprises one or both of a primary analysis or a secondary analysis of the subject's genetic composition; and generating a variant details summary for display or printout, wherein the variant details summary integrates and concurrently shows data, the data comprising: external variant detail data acquired from one or more data stores external to a machine executing the machine-readable instructions; and local variant detail data comprising past case data of a user of the machine or an organization to which the user belongs.

In an additional embodiment, one or more computer readable media are provided comprising machine-executable instructions. The machine-executable instructions, when executed, cause acts to be performed comprising: receiving as a first input a nucleic acid sequence dataset, wherein the nucleic acid sequence data set is an output of one or both of a primary analysis or a secondary analysis; displaying a selectable listing of one or more variants identified in the nucleic acid sequence dataset; receiving a selection of a variant of interest from the selectable listing of the one or more variants; accessing two or more external data stores comprising variant data associated with the selected variant of interest; displaying one or more variant findings accessed from the two or more external data stores; receiving a selection of one or more of the variant findings; creating an assertion for the variant of interest for each selection of the one or more variant findings; and generating a customized report based on the assertions.

In another embodiment, one or more computer readable media are provided comprising machine-executable instructions. The machine-executable instructions, when executed, cause acts to be performed comprising: receiving as a first input a nucleic acid sequence dataset, wherein the nucleic acid sequence data set is an output of one or both of a primary analysis or a secondary analysis; displaying a selectable listing of one or more variants identified in the nucleic acid sequence dataset; receiving a selection of a variant of interest from the selectable listing of the one or more variants; accessing past case data comprising variant data associated with the selected variant of interest; displaying one or more variant findings accessed from the past case data; receiving a selection of one or more of the variant findings; creating an assertion for the variant of interest for each selection of the one or more variant findings; and generating a customized report based on the assertions.

In a further embodiment, one or more computer readable media are provided comprising machine-executable instructions. The machine-executable instructions, when executed, cause acts to be performed comprising: accessing or receiving a data file comprising genetic data for a subject, wherein the genetic data comprises one or both of a primary analysis or a secondary analysis of the subject's genetic composition; and displaying a variant details summary interface, wherein the variant details summary interface integrates and concurrently shows data comprising: external variant detail data acquired from one or more data stores external to a machine executing the machine-readable instructions; and local variant detail data comprising past case data of a user of the machine or an organization to which the user belongs; and providing on or via the variant details summary interface selectable options for creating one or more assertions based on the external variant detail data, the local variant detail data, or a de novo assertion entry.

### BRIEF DESCRIPTION OF THE DRAWINGS

These and other features, aspects, and advantages of the present invention will become better understood when the following detailed description is read with reference to the accompanying drawings, in which like characters represent like parts throughout the drawings, wherein:

FIG. 1 illustrates a sample process flow of a tertiary analysis of genetic data through report generation using a software platform in accordance with aspects of the present disclosure;

FIG. 2 depicts a sample process flow of sequence data acquisition and processing in conjunction with certain technical features used in the process flow, in accordance with aspects of the present disclosure;

FIG. 3 depicts aspects of a software architecture for sequence data processing, in accordance with aspects of the present disclosure;

FIG. 4 depicts a data flow using the software architecture of FIG. 3 or a similar architecture of a software platform for sequence data analysis, in accordance with aspects of the present disclosure;

FIG. 5 depicts a decomposition of modules and components that may be present in a software platform for sequence analysis, in accordance with aspects of the present disclosure;

FIG. 6 depicts a logical architecture corresponding to function for a software platform for performing sequence data analysis, in accordance with aspects of the present disclosure;

FIG. 7 depicts a schematic view of the use of a field of a graphical user interface to specify a folder or location for monitoring for primary or secondary analysis results for tertiary processing, in accordance with aspects of the present disclosure;

FIG. 8 depicts an example of an interface screen of a software platform in which an option to create or modify a personalized knowledge base is displayed, in accordance with aspects of the present disclosure;

FIG. 9 depicts an example of an interface screen of a software platform which may be used for accessing a template and uploading a completed template for populating a personalized knowledge base, in accordance with aspects of the present disclosure;

FIG. 10 depicts an example of an interface screen of a software platform in which an upload status of a template for populating a personalized knowledge base is displayed, in accordance with aspects of the present disclosure;

FIG. 11 depicts an example of an interface screen of a software platform in which cases are listed and which allows a user to select a case for processing, in accordance with aspects of the present disclosure;

FIG. 12 depicts an example of an interface screen of a software platform in which an overview of a selected case is displayed, in accordance with aspects of the present disclosure;

FIG. 13 depicts an example of an interface screen of a software platform in which a listing of variants for a selected case is displayed and from which a variant may be selected for interpretation and visualization, in accordance with aspects of the present disclosure;

FIG. 14 depicts an example of an interface screen of a software platform in which a listing of variants for a selected case is displayed along with an indication of the presence of personalized knowledge base content relevant to a respective variant, in accordance with aspects of the present disclosure;

FIG. 15 depicts an example of an interface screen of a software platform in which personalized knowledge base data for a variant is displayed, in accordance with aspects of the present disclosure;

FIG. 16 depicts an example of an interface screen of a software platform in which a variant of a case has been selected and relevant past case data is displayed, in accordance with aspects of the present disclosure;

FIG. 17 depicts an example of an interface screen of a software platform in which a relevant past case has been selected for a variant under review to form an assertion, in accordance with aspects of the present disclosure;

FIG. 18 depicts an example of an interface screen of a software platform in which a de novo assertion has been created and added for a variant under review to form an assertion, in accordance with aspects of the present disclosure;

FIGS. 19A and 19B in combination depict an example of an interface screen of a software platform in which an assertion is illustrated along with past case, knowledge base, and clinical trial findings for a variant under review, in accordance with aspects of the present disclosure;

FIG. 20 depicts an example of an interface screen of a software platform in which an overview of variant information is displayed for a variant under review, in accordance with aspects of the present disclosure;

FIG. 21 depicts an example of an interface screen of a software platform in which an overview of gene information is displayed for a variant under review, in accordance with aspects of the present disclosure;

FIG. 22 depicts an example of an interface screen of a software platform in which predictors are displayed for a variant under review, in accordance with aspects of the present disclosure;

FIG. 23 depicts an example of an interface screen of a software platform in which population data is displayed for a variant under review, in accordance with aspects of the present disclosure;

FIG. 24 depicts an example of an interface screen of a software platform in which a case detail overview is provided with biomarker interpretation data, in accordance with aspects of the present disclosure;

FIG. 25 depicts an example of an interface screen of a software platform in which biomarker data, here tumor mutational burden, is displayed, in accordance with aspects of the present disclosure;

FIG. 26 depicts an example of an interface screen of a software platform in which biomarker data, here microsatellite instability, is displayed, in accordance with aspects of the present disclosure;

FIG. 27 depicts an example of an interface screen of a software platform in which biomarker data, here genomic instability score, is displayed, in accordance with aspects of the present disclosure;

FIG. 28 depicts an example of an interface screen of a software platform in which a filter may be configured or otherwise specified, in accordance with aspects of the present disclosure;

FIG. 29 depicts an example of an interface screen of a software platform in which a draft report may be configured and/or reviewed, in accordance with aspects of the present disclosure;

FIG. 30 depicts an example of an interface screen of a software platform in which a further example of a draft report may be configured and/or reviewed, in accordance with aspects of the present disclosure;

FIGS. 31A and 31B in combination depict an example of a report, in accordance with aspects of the present disclosure; and

FIG. 32 depicts an example of an alternative report in a different application context, in accordance with aspects of the present disclosure.

### DETAILED DESCRIPTION

Methods and systems described herein relate to the configuration and use of a customizable software platform capable of receiving a nucleic acid sequencer data file or variant file as an input and that provides tools for users to perform analysis (e.g., tertiary analysis) of the raw or previously processed sequence data. As used herein, a software platform may be understood to comprise processor-executable code or routines (or other machine-executable code or routines) stored and accessible from a memory or storage medium and which, when executed by a processor, performs actions (or otherwise provides functionality) as described herein in the context of sequence and variant processing. As further described herein, such a software platform may be implemented locally or on-site (e.g., on-premise) or in a distributed manner in which local resources (e.g., a workstation and browser) communicate with and interact with a cloud platform to cooperatively implement the code and functionality of the software platform as described herein. The sequence data file provided as inputs to the software platform may be generated using a next-generation sequencing (NGS) device and in practice may be human or non-human (e.g., viral, microbial, animal origin, plant origin, and so forth) DNA or other nucleic acid samples. In certain embodiments, the sequence data in question may have undergone primary and secondary analysis. As used herein, primary data analysis may be understood to be an analysis (such as may be performed using processor or hardware implemented algorithmic steps) that operates during cycles of sequencing chemistry and imaging and which provides base calls and associated quality scores representing the primary structure of DNA or RNA strands. An output of such a primary analysis may, for example, be a. FASTA file, which is a text file containing nucleotide sequence data represented in single-letter codes and the associated quality scores. Further, as used herein a secondary data analysis may be understood to be an analysis (such as may be performed using processor or hardware implemented algorithmic steps) that performs alignment and assembly of DNA or RNA fragments to provide the full sequence for a nucleic acid sample, from which genetic variants can be determined. Such a secondary analysis may be performed with reference to a reference genome for calling sequence variants and imputing genotypes. Additionally, a secondary analysis may calculate tumor mutational burden (TMB), microsatellite instability (MSI), or genomic instability score (GIS). An output of such a secondary analysis may, for example, be a variant, call file (VCF). In addition, as used herein tertiary data analysis (such as may be performed using processor or hardware implemented algorithmic steps) may employ biological data mining and interpretation tools to obtain useful insights based on the primary and secondary analysis results, such as by facilitating the interpretation of genetic variation to obtain knowledge and insights into basic biology, causes of diseases, and/or treatment options. By way of example, such analytics may be useful in determining links between observed variant data and an observed phenotype in a patient. With the preceding in mind, a software platform as discussed herein may receive the output(s) of primary and/or secondary analysis as an input file (e.g., a generic or standardized input file, such as a VCF or FASTA file) in order to facilitate tertiary analysis and user interpretation and reporting of the results of such analysis.

In practice, the software platform may access one or more internal and/or external data stores (e.g., genetic variant knowledge bases, including but not limited to public, commercial, government, and academic databases as well as past case data of the user or their organization) and the software platform may provide or display relevant information obtained from the data stores to the user as part of the operation of the software platform. For example, the software platform may function as generic middle ware that allows users to store, arrange, annotate, and/or visualize genetic data (e.g., sequence or variant data) with information available from separate or external databases and/or from internal or past case datafiles or databases. The user may in turn configure or prepare a customized report using the software platform and selected information obtained from the data stores. In this manner the software platform may provide, via one or a series of interfaces, genotype information, phenotype information, and/or clinical information for use by the user in generating a customized report.

It may also be appreciated that the present software platform may be employed in a variety of sequence analysis applications including, but not limited to oncology testing, environmental surveillance (metagenomics), anti-microbial resistance (AMR) studies, infectious disease studies, public health and microbial surveillance (e.g., viral lineage studies), genetic disorder studies, genetic disease testing (including detection of rare undiagnosed genetic diseases (RUGD)), and so forth. By way of example, in the context of genetic disease testing, the presently described software platform may help automate and provide efficiency gains and cost reduction in such testing, including carrier screening and accelerated evidence generation related to RUGD. In the context of oncology testing, the presently described software platform may facilitate automation, customization, and selection of relevant third-party or internal knowledge bases to simplify reporting and therapy selection, and so forth. In the context of infectious disease studies, the presently described software platform may facilitate implementation of user-defined viral and/or microbial lineage and Glade assignment via access to relevant third-party knowledge bases.

In practice, the context or application relevant to the sequence analysis may determine the data sources accessed as part of the review and interpretation process. That is, in an oncology context, databases relevant to oncology may be accessed for relevant data while in an infectious disease study context data sources relevant to infectious diseases are accessed. In certain embodiments the software platform facilitates user review and custom report generation but does not itself analyze or interpret genetic data. For example, the software platform may make relevant data available from accessed internal and/or external data sources in a standardized interface for user review, interpretation, annotation, and selection of which data to include in a generated custom report, which typically will pertain to genetic variants found in the sequence data. In other embodiments the software platform may provide some element of automated analysis or interpretation of the genetic data to facilitate user review and custom report generation.

Configurability of the software platform may include features which allow a user or an organization to define custom workflows, which may be specific to a user or group of users, to an application (e.g., oncology, infectious disease and microbial surveillance (such as viral and/or microbial lineage), genetic disease testing (such as rare undiagnosed genetic diseases (RUGD) testing, carrier screening, pharmacogenomics, and so forth)), and/or to sample source or sequencing technique (e.g., panel-based sequencing, whole genome sequencing, whole transcriptome sequencing, whole exome sequencing, DNA, RNA, tumor-only, tumor/normal tissue mixed, solid, heme, circulating tumor DNA (ctDNA), and so forth), and/or to a sample type. Further, aspects of the workflow associated with the software platform may be configurable or customized based on user or organization preference or procedure (e.g., standard operating procedure (SOP)). In certain implementations the software platform may be integrated with or otherwise in communication with a laboratory information management system (LIMS) and/or electronic health record system (EHR).

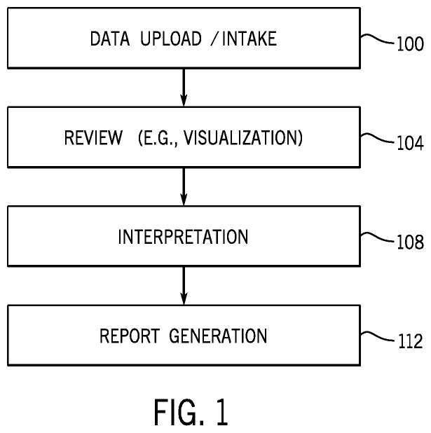

By way of example, and to provide real-world context, an implementation of a workflow based on the presently described software platform is provided. In accordance with this implementation, and with reference to FIG. 1, an initial step in a workflow may involve case accessioning, such as receiving and intaking (block 100) a new case in the form of a FASTA or VCF file subsequent to a sequencing operation. Based on a standard operating procedure (SOP) for the user or organization, the new case may undergo variant review, filtering, and prioritization. The software platform may simplify such a variant review step (block 104) by performing or facilitating a quality control operation related to variant identification and/or variant quality metrics, providing visualizations (e.g., a genomics viewer) related to the identified variants and/or filtered variants based on user selections, and/or providing frequency information (e.g., population or sub-population frequency information, demographic frequency information, and so forth) related to the variants in question. Subsequent to variant review, the software platform may facilitate a variant interpretation operation or step (block 108), such as may be performed in accordance with the user or organizations SOP. As used herein “interpretation” or “variant interpretation” may be understood to include or involve the association of information to variants to inform functional and/or clinical significance of the variants. By way of example, the software platform may access one or more of external (e.g., third-party databases) or internal knowledge bases based on the present context or application (e.g., oncology, infectious disease, microbial surveillance, genetic disorders, and so forth), may access relevant past case data for one or more of the variant(s) being reviewed (e.g., historic or prior decisions) for the user or organization, and/or may access functional impact data from relevant data sources for association with the variant(s) being reviewed. Such information may be provided to and used by the user of the software platform to annotate the sequence or variant data, such as to make associations between the identified variants and the information in the accessed databases. As used herein, “annotation” or “variant annotation” may be understood to include or involve providing variant characteristics related to the variant's structure, functional impact, prevalence, potential clinical impacts, and so forth. Based upon the variant interpretation step, the software platform may further facilitate user generation, modification, and approval of a report (block 112) relevant to the case. Aspects of the report, such as but not limited to layout, annotations, and other presentation aspects, may be configurable by the user or their organization so as to facilitate generation of customized reports. The software platform may further facilitate final approval or sign-out of the report once reviewed and approved.

While certain implementations of the software platform as described herein may be local or on-premise, in other implementations the software platform may be implemented as part of a cloud-based platform or architecture (e.g., a multi-regional, multi-tenant cloud deployment). By way of example, a software platform as discussed herein may be integrated with an independent computing architecture (ICA) for large scale data warehousing and cohort analysis. In such an implementation, an infrastructure may be provided, as discussed in greater detail below, to support data upload to the ICA and to process data from any ICA project.

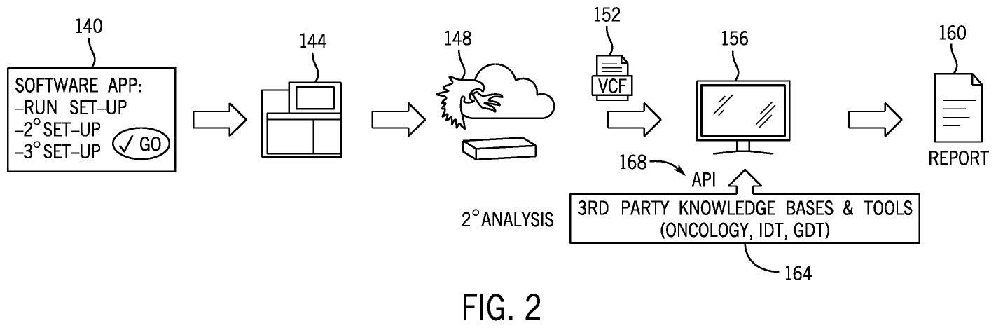

With this in mind, FIGS. 2-6 depict various aspects of a computer-based implementation of the presently described software platform, including aspects that may be present in an on-premise implementation as well as features that may be specific to a cloud-based implementation. By way of example, and turning to FIG. 2, a high-level overview of a technical implementation is illustrated. In this example a management application 140 may be employed to set up or parameterize a nucleic acid sequencer run, to set up or specify a secondary analysis of the sequencer output, and/or to set up or specify a tertiary analysis of the secondary analysis output. Once such sequencer parameters and/or analytics are specified, the nucleic acid sequencer 144 may perform a sequencing operation on a sample in accordance with the specified parameters. The sequence output from the sequencer 144 may undergo secondary analysis 148 using either propriety or internal analytics and/or external or third-party analytics. Results of the secondary analysis may be output as a variant call file (VCF) 152, which in the depicted example is in turn an input to a software platform 156 that may facilitate tertiary analysis of the data as well as generation of a report 160 of the results. As shown in this high-level overview and as discussed elsewhere herein, the software platform 156, implemented as either an on-premise or cloud-based implementation, may access one or more third-party resources 164 (e.g., knowledge bases and/or tools which may be specific to a particular application (e.g., oncology, infectious disease testing, genetic disease testing, and so forth)) relevant to the analysis. As shown in this example, one or more application programming interfaces (APIs) 168 may be employed to facilitate interactions between the software platform 156 and the third-party (i.e., external) resources 164.

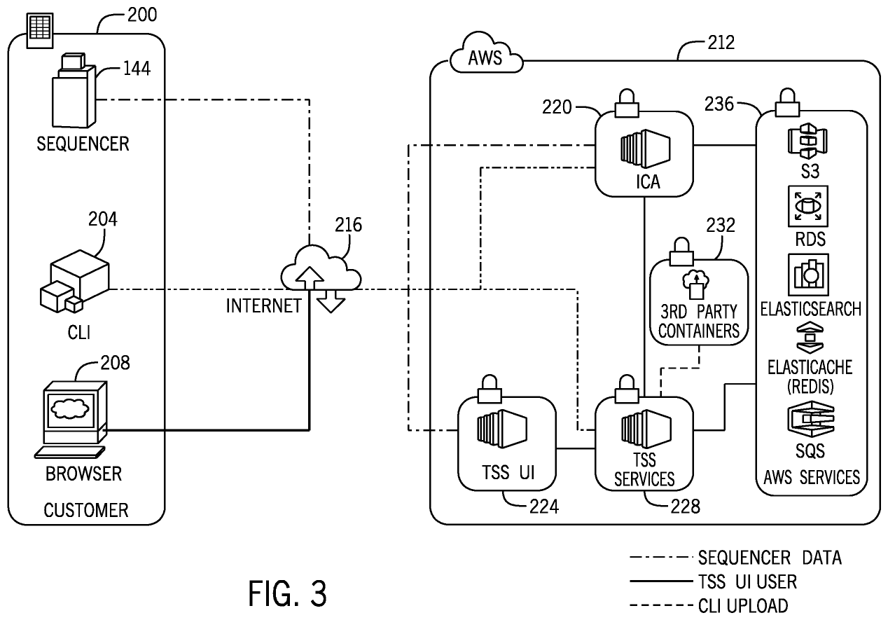

With this high-level overview in mind, and turning to FIG. 3, an example, of a cloud system architecture in which the software platform 156 may be implemented is provided. In accordance with this example, a client site 200 is depicted at which aspects of the presently disclosed approach are depicted as being present or deployed. For example, the client site 200 may include one or more nucleic acid sequencers 144, a command line interface (CLI) 204 for data uploads, and one or more computers 208 suitable for running a web browser capable of communicating with a cloud platform 212 (here depicted as an Amazon Web Services (AWS) data center) over the internet 216 or other network. Executable code and routines associated with the software platform 156 (as well as data with which the software platform 156 interacts) may be stored, updated, and implemented at the cloud platform 212. In the depicted example, the cloud platform 212 encompasses an independent computing architecture (ICA) 220, a TSS user interface (UI) component 224, a TSS services component 228, third-party containers 232, and cloud platform services 236, some or all of which may be involved in implementing the software platform 156.

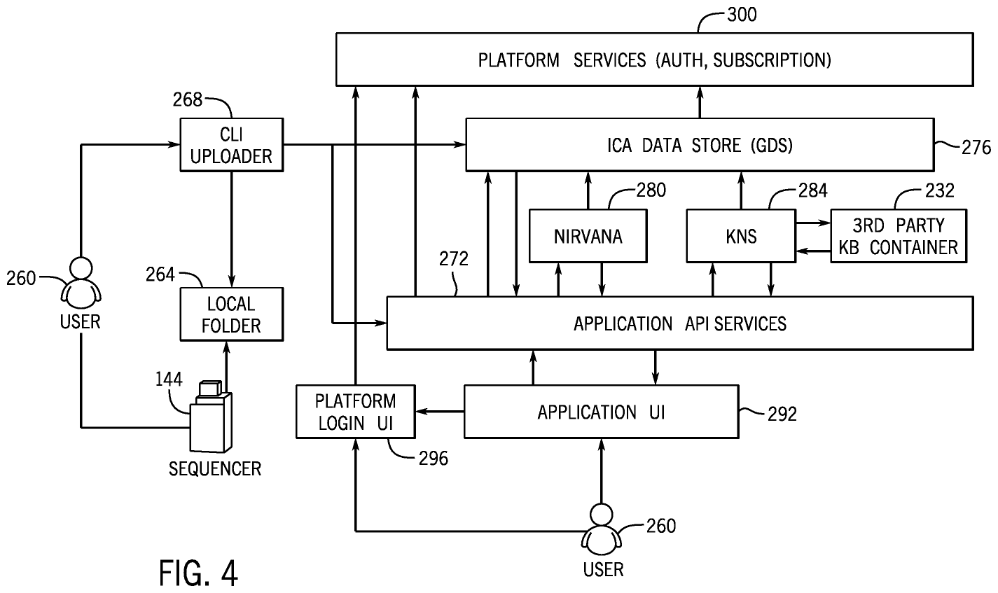

Turning to FIG. 4, an example of a data flow based on the presently described software platform 156 and cloud platform 212 is depicted. In accordance with the depicted example data flow, a user 260 may configure aspects of the systems as a preliminary matter, such as configuring the domain, users, and/or parameters or other customizable features of the software platform 156. Once system configuration has been performed, a sequencing run may be performed using the sequencing instrument 144, which may output a results file (e.g., a FASTA or VCF file) to a local folder 264. In the depicted example, the user 260 may then launch a CLI uploader 268 to upload the results file to the software platform 156. In the depicted example, the CLI uploader 268 communicates via the application platform API 272 to create a case and to cause the data from the file to be uploaded to an ICA data store 276 which may be a repository suitable for short-term or long-term data storage of genetic data. The application platform services, via the application API 272 reads or otherwise accesses the data from the ICA data store 276 to initiate tertiary analysis processing. In the depicted example, the data being processed (e.g., VCF data) is annotated, such as using a service 280 such as Nirvana which may receive a VCF file and output a structured JSON representation of some or all annotation and sample information extracted from the VCF. Via a knowledge network service (KNS) 284 of the application platform 156, which may access third-party (or internal) knowledge base containers 232, knowledge base annotations are processed for the case. The user 260 (or a different user) may log into the software platform 156, such as via a platform login user interface (UI) 296 configured to communicate with platform services 300 (which may provide authentication and/or subscription services) when the case is ready for interpretation (e.g., once knowledge bases have been accessed and the case annotated). The user may then interpret the case as discussed herein using an application UI 292.

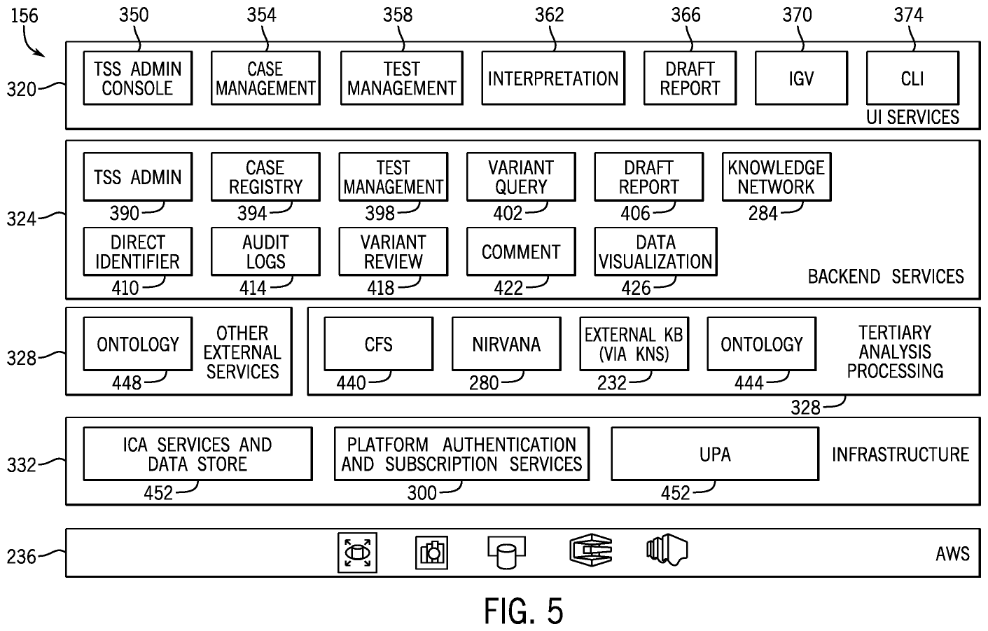

Turning to FIG. 5, a decomposition of one implementation of the software platform 156 is illustrated in the context of a high-level block figure. As shown in this example, the software platform may comprise modules or components corresponding to UI services 320, backend services 324, tertiary analysis processing 328, infrastructure 332, other external services 336 and cloud platform 236 (in a cloud-based implementation). In the depicted example, the UI services 320 of the software platform 156 may include some or all of a TSS admin console 350, a case management interface or module 354, a test management interface or module 358, an interpretation interface or module 362, a draft report interface or module 366, a genomics viewer interface or module 370, and/or a CLI interface or module 374. The backend services 324 of the software platform 156 may include some or all of a TSS admin module 390, a case registry module 394, a test management module 398, a variant query module 402, a draft report module 406, a knowledge network module 284, a direct identifier module 410, an audit logs module 414, a variant review module 418, a comment module 422, and/or a data visualization module 426. The tertiary analysis process modules or components 328 of the software platform 156 may include some or all of a common fragile sites (CFS) module 440, an annotation service 280 (such as Nirvana), an external knowledge base module 232, an ontology module 444, and so forth. Similarly, the other external services 336 accessed by or incorporated in the software platform 156 may include an ontology module 448 or similar functionality provide by an external service or site. In addition, the infrastructure modules or components 332 of the software platform 156 may include some or all of ICA services and data store 452, platform authentication and subscription services 300, and/or a UPA module 452.

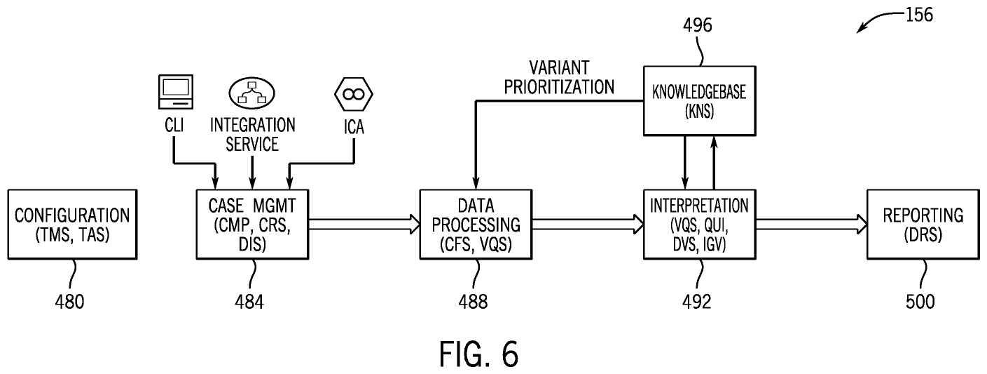

Turning to FIG. 6, an alternative view of workflow using the software platform 156 described herein is presented. In the depicted system architecture, aspects of the software platform are characterized by functionality, such as configuration 480, case management 484, data processing 488, interpretation 492, knowledge bases 496, and reporting 500. Configuration functionality 480 may include, but is not limited to knowledge base selection, test configuration, column configuration, and report configuration. By way of example column configuration may include specifying a normalized (e.g., common or shared) column and/or field layout for data from different knowledge bases to be presented so as to facilitate review and comparison. With respect to case management functionality 484, this may include, but is not limited to, supporting case data upload via multiple techniques, such as on-premise upload via CLI, upload of secondary analysis results via ICA, and/or obtaining data via an integration service. Cases may be managed or supported for files by application type (e.g., oncology applications may support VCF files, quality control (QC) results, and/or a sample sheet, infectious disease testing may support FASTA files, and so forth). Such case management functionality may be implemented as a portal by which a user may create new cases, import genetic data files, and/or enter meta-data associated with a case. Data processing functionality 488 may include, but is not limited to, annotation using knowledge bases, such as an annotation service or module (e.g., Nirvana), one or more knowledge network services (KNS) related to a selected application (e.g., oncology, infectious disease testing, genetic disease testing, and so forth), custom scripts, initialization of downstream services, and/or preparation of an interpretation view. Interpretation functionality 492 may include, but is not limited to, case details, a case overview or summary, variant details, knowledge base associations, past case data, genomics viewer-based tool visualization, and/or quality control (QC) summary. Knowledge base functionality 496 may include, but is not limited to, association for all variant types, bulk upload, version support, external knowledge base integration, and/or a pluggable knowledge base architecture. In addition, reporting functionality 500 may include, but is not limited to, generation and/or use of customizable templates, report signoff or approval, editing (e.g., addend or amend) of reports, and/or support of suitable formats (e.g., JSON, PDF, and so forth).

As discussed herein, and with the preceding architectural details and examples in mind, the presently described software platform 156 allows users to store, arrange, and visualize human or non-human (e.g., viral, microbial, animal origin, plant origin) next-generation sequencing (NGS) data. In addition, the software platform 156 allows users to view content from appropriate external sources, such as based on the application or use-case). Alternatively or in addition, the software platform 156 may also provide or display content or data related to past cases of the user or their organization, which may allow the user to quickly review if their previous work is relevant to the case they are analyzing. The content of such data sources provided for view or consideration may be filtered by the user to define relevant content, such that only such content is displayed for the user to consider.

In view of the data provided for review by the user, the user may annotate the genetic data with the information available from the accessed genetic databases (e.g., external (i.e., third-party)) or past cases and to create custom reports. These separate genetic databases accessed by the software platform 156 may, as discussed herein, cover use-case applications such as infectious disease testing, oncology, microbial surveillance, genetic disorder testing and so forth and may allow users to incorporate or review available information from public, commercial, governmental, or academic databases as well as users' internal genetic databases. Such functionalities allow the user to make meaningful associations between information contained in a genetic data input file and information in one or more relevant databases. As discussed herein, such databases may be relevant to infectious diseases, oncology, microbial surveillance, genetic disorders, and so forth. Additionally, the software platform 156 as presently described allows a user to customize the content of a report based on their selected findings by populating sections of the report pertaining to specific variants present in the sample. In certain implementations the user may edit the content of the knowledge bases as presented in the report so as to be applicable to the sample or subject or may provide their own de novo interpretation.

As discussed herein, and as illustrated by representative screenshots in the following discussion, a sample workflow using the software platform 156 may include steps or procedures for case initiation. For example, to process a case a user may first select an application (i.e., use-case) of interest relevant to a respective nucleic acid sample. In practice, this may involve selecting an application of interest (e.g., environmental surveillance (metagenomics), oncology testing, anti-microbial resistance (AMR) study, viral lineage study, genetic disorder study, and so forth) from one or more selectable options provided on a user interface (UI).

In a further aspect, the user may select options within the software platform 156 to upload the sequence and/or variant information (e.g., a VCF or FASTA file) relevant to the selected application. As noted herein, a command line interface (CLI) or CLI uploader may be utilized as part of the process of uploading sequence or variant information for processing using the software platform 156. In certain implementations, however, a graphical user interface may be provided to a user which allows the user to specify a file or directory location for monitoring by the software application 156. In such cases, when a new sequence listing or variant file is detected in the target folder 264 or directory, the CLI uploader may be automatically triggered to upload the detected file for processing as described herein.

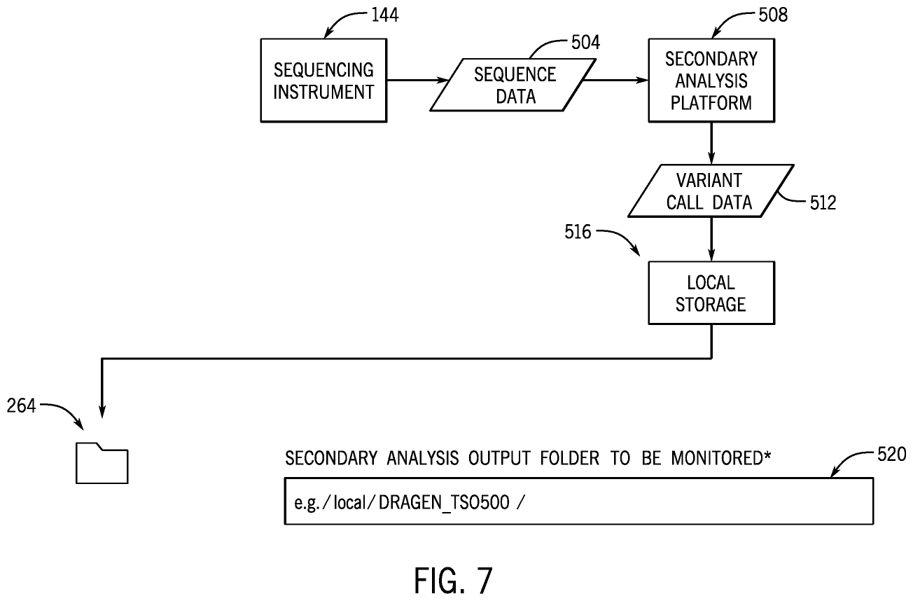

By way of example, and turning to FIG. 7, a schematic view is depicted of such a flow. In this example, a nucleic acid sequencer 144 is illustrated as generating sequence data 504. The sequence data 504 may automatically undergo secondary analysis for variant calling, such as via a suitable secondary analysis platform 508 or program. By way of example, FIG. 7 depicts the receipt of the primary sequence data 504 by a Dynamic Read Analysis for GENomics platform (e.g., a DRAGEN® platform 508, provided by Illumina, Inc.), which in turn may automatically output a variant call file 512 or corresponding secondary analysis output. In the depicted example, the variant call file 512 (or sequence data 504 in embodiments in which the software platform 156 can processes the primary analysis data) is stored to a local folder 264 within local storage 516. As also shown in FIG. 7, an example of a user-fillable field 520, provided as part of a graphical user interface (GUI), may be provided as part of the software platform 156 and may be used by a user to specify a path or location of the local folder 264 for periodic or continuous monitoring. As files (e.g., sequence data, variant call files, test definitions) are added to the monitored location specified by the field 520, the CLI uploader (or comparable automated upload functionality) may upload the data for processing by the software platform 156, as discussed herein.

With the preceding in mind, once the user has selected an application and uploaded or otherwise accessed the relevant sequence or variant data, the user, using the software platform 156 may arrange the list of variants for review by filtering and/or sorting through a set of customized filters. By way of example, the software platform 156 may provide the user with an interface and tool (i.e., a variant filtering tool) by which the user may select, configure, and apply filter criteria and/or sort genetic data for review. Example filtering conditions may include, but are not limited to, variant genomic position, variant allele frequency, variant population frequencies from selected databases, variant type, quality metrics, variants in a gene on a user-configured gene-list, and so forth.

The software platform 156 may further provide one or more tools for visualizing some or all of the variants present in the genetic data. By way of example, such a variant visualization tool may allow the user to visualize and inspect genomic data (including read alignments) at the variant, gene, chromosome, or whole genome levels. An example of a suitable visualization tool may be, but is not limited to, a genomics viewer tool or similar visualization tool, which may be configured to allow the user to inspect genomic data, such as read alignments. In addition to variant-level visualizations, a genomics viewer tool provided as part of the software platform 156 may provide views of an entire chromosome or whole genome that allows the user to look for large anomalies.

The software platform 156 may further provide one or more tools for interpreting genetic data. By way of example, and as discussed herein, the software platform 156 may provide an interface for the display, review, selection, and/or editing of variant information derived for the sample or case in question and annotation information from accessed databases or past case data selected or specified by the user. By way of example, in accordance with aspects of the software platform 156 as described herein, relevant genetic information for a case, selected by the user, may be aggregated on an interface for review. The information may include variant annotations from one or multiple genetic databases for the specific application (i.e., use case). For example, in the context of a genetic disease study application, the user may choose to display annotations from databases such as ClinVar, OMIM, gnomAD, or COSMIC. In the context of an oncology testing application, the user may choose to display annotations information from databases such as PierianDx, OncoKB, or CKB. In the context of a pathogen lineage and microbial research application, the user may choose to display information from Nextclade, Pangolin, or AMRfinder.

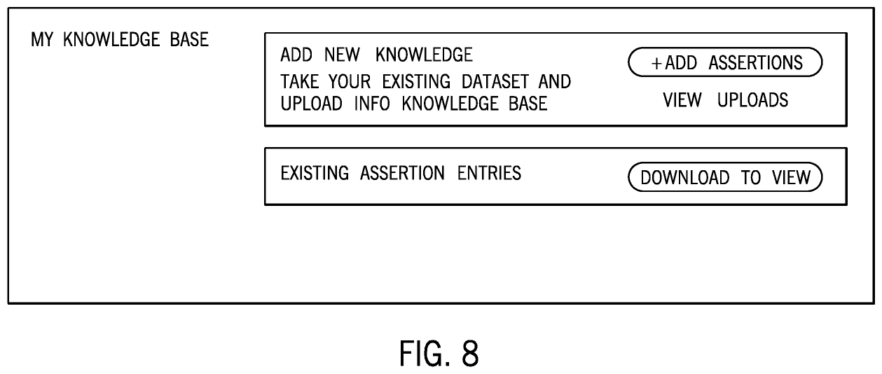

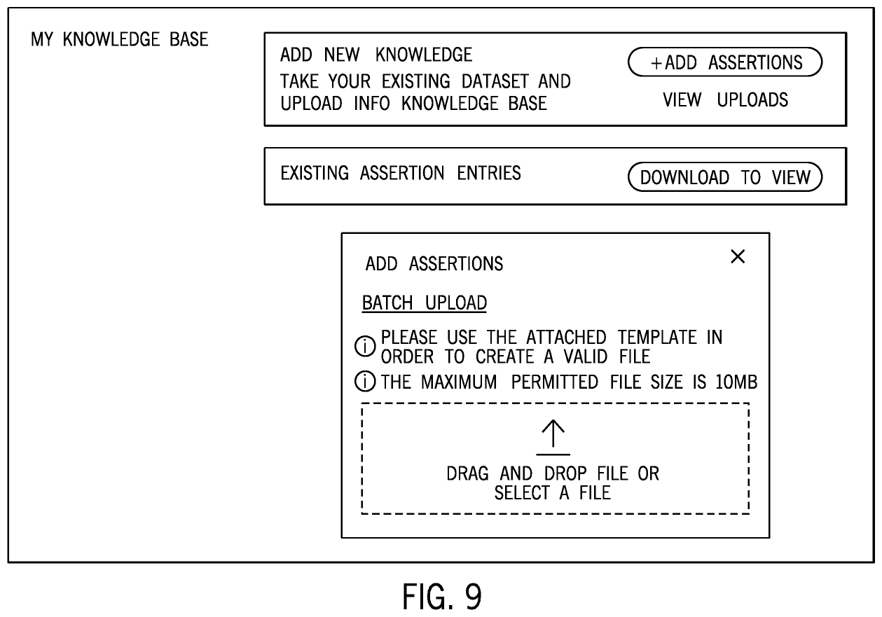

Further, past case data (i.e., interpretations of genetic data from the user's laboratory and other laboratories (e.g., “crowd sourced”) may also be displayed and used to inform the user's interpretation of the current case. By way of example, and turning to FIGS. 8-10, examples of sample GUI screens are provided illustrating steps by which a user may add or update past case data and/or individual or organization preferences to a personalized knowledge base (e.g., “My Knowledge Base”) for inclusion during processing of sequence or variant data. As will be appreciated, in accordance with this approach a user may re-use prior data not only from the current software platform 156, but also from other or prior systems or historical data. In this manner, the software platform 156 may be pre-loaded with a user's or organization's prior work or preferences and may incorporate such work or preferences without additional training and from initial use of the platform 156. In the depicted example screens, and turning to FIG. 8, a user may be presented with an option add entries or “new knowledge” to a personalized knowledge base. In this example, and turning to FIG. 9, selecting the “+ Add Assertions” option results in the GUI displaying an additional interface element to facilitate an upload process (e.g., a batch upload) via a drag-and-drop or file selection input mechanism.

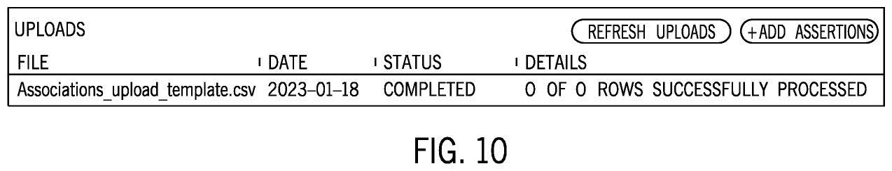

In certain embodiments the file or data to be uploaded may differ in format from what is suitable for the software platform 156. By way of example, the data to be uploaded may have additional data columns, may be missing expected columns, may employ different column names, and/or may have columns if a different order than what is expected by the software platform 156. With this in mind, it may be useful to reformat the data to be uploaded either prior to the upload process or as part of the upload process. To facilitate such data importing, therefore, the illustrated interface element displayed in response to a user selecting to “+ Add Assertion” provides an option to download or otherwise access a template (e.g., a CSV file) that may be populated with the data to be uploaded so that such data is in a suitable format for uploading. In the depicted example screen, the populated template may be dragged-and-dropped onto an upload region of the interface or otherwise selected for upload. Turning to FIG. 10, an example of a screen is provided that depicts the upload status and details of a selected template file (here depicted as a template CSV file).

In practice data derived from multiple data source for a given application may have different fields and/or layouts, the software platform 156 described herein may, to facilitate review and comparison, impose a normalized (e.g., common or shared) layout on displayed data (e.g., mapping data fields or columns to the normalized layout) to as to facilitate user review and consideration of the displayed data. As part of the interpretation process, the user can select and include certain genetic information (via an interface of the software platform 156) such as particular sequence variants and their interpretations for inclusion in a report. This selection process, as used herein, may be referred to as an “assertion”. Via the review and interpretation interface(s) of the software platform 156, a user may choose to add certain genetic information and interpretations to a report by creating such an assertion.

With respect to report generation, editing, and approval via the software platform 156, as a user completes variant interpretation for a case by creating one or multiple assertions, a report (e.g., a PDF or JSON report) may be created by the software platform 156 for user approval or sign out. Further, the user can customize the format (e.g., layout) of the report to include the organizations logo or name as well as relevant comments.

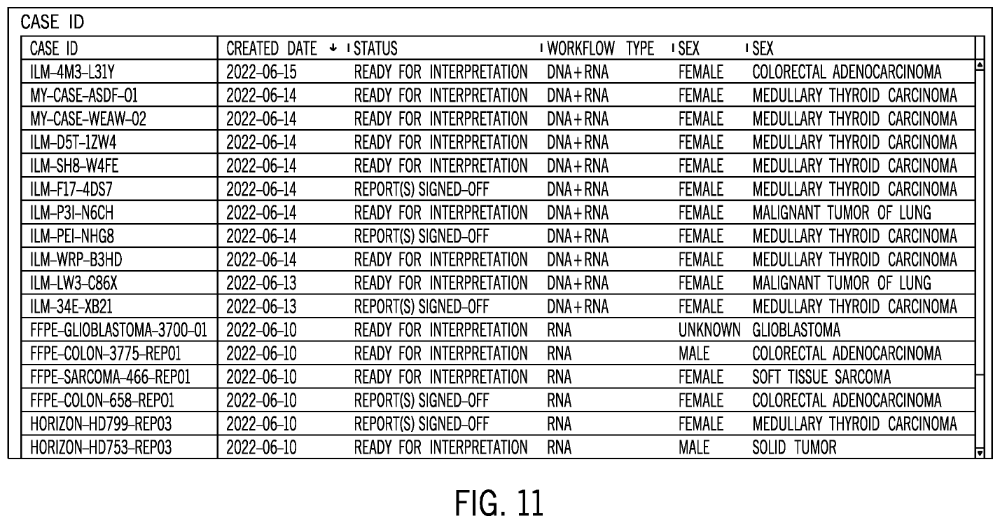

With the preceding in mind, FIGS. 11-32 depict sample screen views illustrating certain aspects of the software platform functionality and interfaces as described herein. As will be appreciated, such examples are not intended to be exhaustive or limiting in design or form, and are instead merely provided to illustrate certain described concepts in a non-limiting manner so as to provide a real-world context and to facilitate understanding of the concepts discussed herein. By way of example, and turning to FIG. 11, an example of a screen illustrating a case listing and selection screen is provided. As may be appreciated, a user may, via an interface displaying such a screen, select a case for processing.

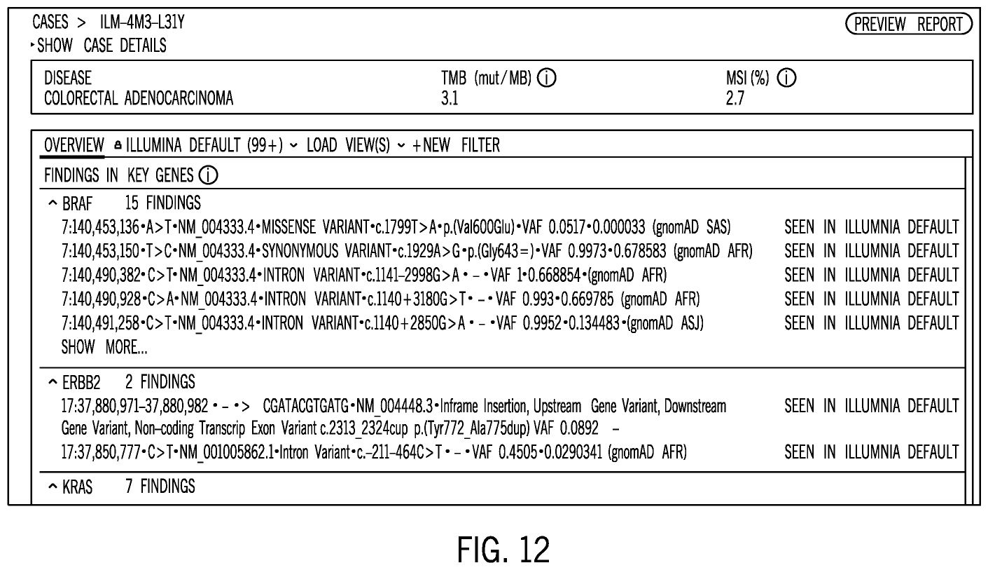

Turning to FIG. 12, upon selection of a case (such as via selection of a case displayed as shown in FIG. 11), a case overview or summary screen may be displayed via the software platform 156. As shown in this example, the case summary includes a disease name along with relevant observed or measured values to that case in question (such as tumor mutation burden (TMB) and microsatellite instability (MSI)). In addition, in a displayed overview tab, key findings are listed with respect to variants identified for the case in question. As shown in this example, interface features are provided to allow a user to expand or collapse categories of findings at the variant level so as to facilitate review.

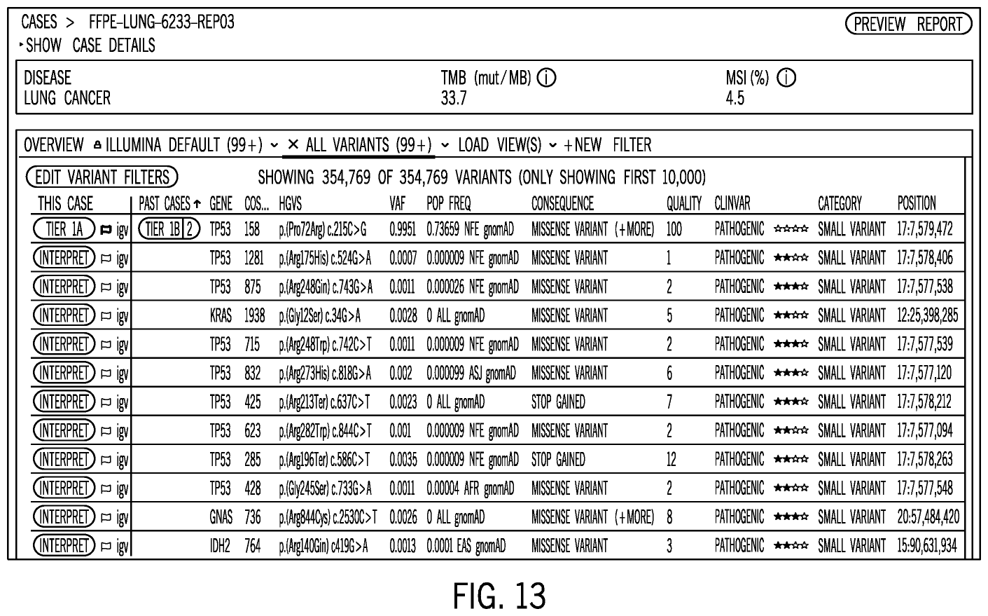

Turning to FIG. 13, an “all variants” view within the case overview interface is depicted. As shown in this view, various user options are provided for flagging and filtering variants of interest. As shown, columns and fields may be provided so as to indicate to a user which variants have been interpreted, and how a characterization or interpretation of the variant, whether a variant is flagged as being of interest or concern, the gene in question in which a variant is present, a variant allele frequency, a population frequency, a consequence associated with the variant, a quality score associated with the read and/or classification, a source (e.g., external database) of the variant data, a category of the variant, and a position of the variant. In the depicted example, an actionability criteria is also displayed for an interpreted case (e.g., “Tier 1A”). This means that the user has opened the “Interpret” button and has assigned an Actionability of Tier 1A. The variant in the top row also has occurred previously in 2 samples at the site (same variant and disease). This is indicated in the Past Cases column, which has a visual for Tier 1B and a count of 2. As used herein, such actionability criteria (Tier 1A, Tier 1B) may be externally defined (e.g., a standardized or conventional actionability criteria) or may be customizable by the user or their organization. In practice, when applied, such actionability criteria may specify a workflow (e.g., treatment or treatment progression) for a given subject having a respective genetic variant or disease.

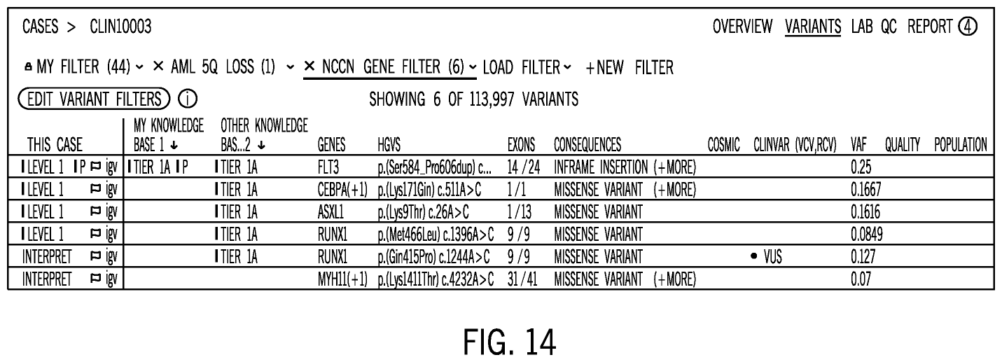

Turning to FIG. 14, a further aspect of data presentation is provided in a context in which a personalized knowledge base (e.g., “My Knowledge Base”) is accessed and utilized in data analysis. As shown in this example, a separate column is provided for results or analytics generated based on the personalized knowledge base relative to other (e.g., external or third-party) knowledge bases. In addition, the provided example also provides columns for the display of the respective gene, a variant descriptor or identifier (e.g., HGVS), an exon identifier or number, a consequence associated with the variant, a quality score associated with the read and/or classification, a source of the variant data, a variant allele frequency, and a population frequency. In the depicted example, actionability criteria are also displayed for interpreted variants (e.g., “Tier 1A”).

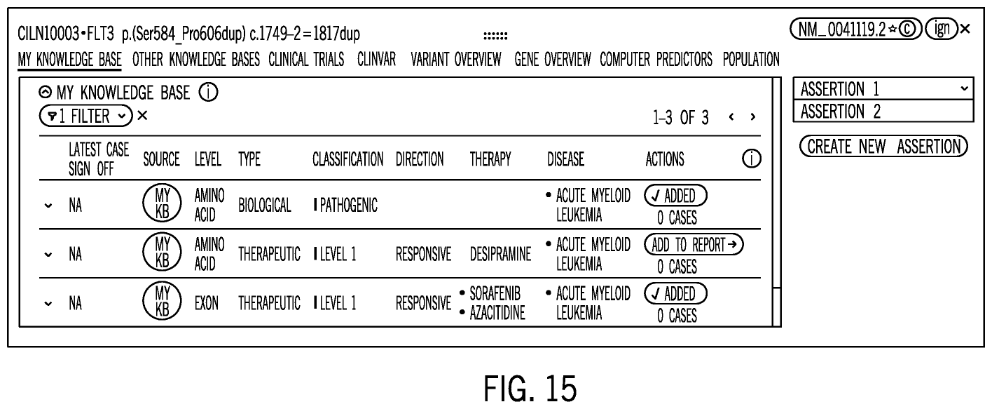

Turning to FIG. 15, in such a personalized knowledge base context, upon selection of a variant (such as via selection of a variant via the interface screen as shown in FIG. 14), a personalized knowledge base overview or summary screen may be displayed via the software platform 156. As shown in this example, the personalized knowledge base results may be chosen for display for the selected variant. In this example, columns are illustrated that correspond to the latest case sign, the data source (the personalized knowledge base or “my KB” in this example), the variant level (e.g., amino acid, exon, and so forth), the information type (e.g., biological, therapeutic, and so forth), a classification, a trend or treatment direction, indicated therapies (if any), disease states or designations, and an action column corresponding to whether data in a given row is to be added as an assertion to the draft or final report. In the depicted example, the first and the third rows have been selected for addition to the report, and are shown as assertions 1 and 2 on the right, while the second row is depicted as still displaying a selectable option to be added to the report. As discussed herein, the personalized knowledge base results illustrated in the manner shown in FIG. 15 may correspond to prior cases handled by the individual (or their organization) compiling the report, and hence reflects information most likely to be relevant to the reviewer in view of their prior experience.

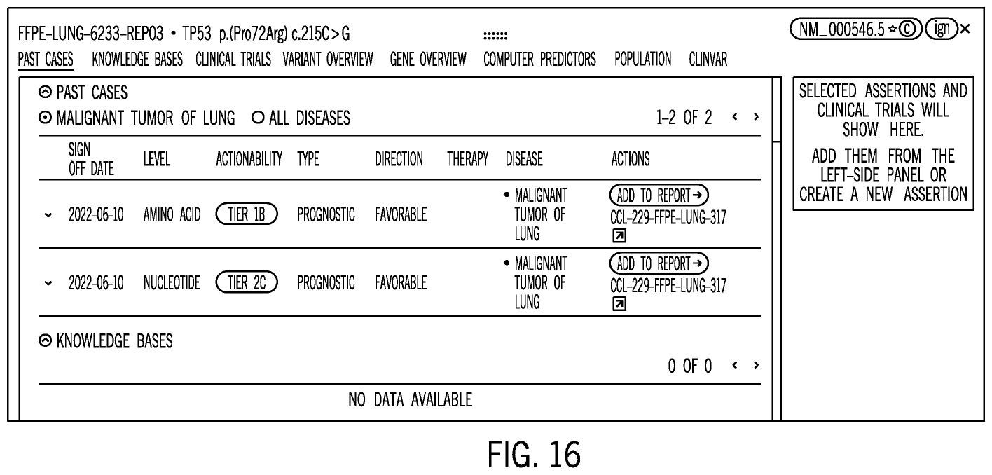

Turning to FIG. 16, by selection of a variant, such as via the screen shown in FIG. 13, a variant detail screen may be displayed. In the depicted example, and as discussed herein, past case data (e.g., past cases of the user or their organization relevant to the selected variant) is illustrated. Data shown in such a context may include data associated with the past case, a level of the variant, an actionability with respect to the variant in the past case as selected by the user who created the assertion in that case, a type (e.g., prognostic, diagnostic, therapeutic, and so forth) of past case indication, a direction associated with the past case (e.g., favorable or unfavorable), indicated therapy (if any), the disease, and an action option for the user which provides an option to the user to add one or more of the past case assertion(s) to the report being generated for the present case.

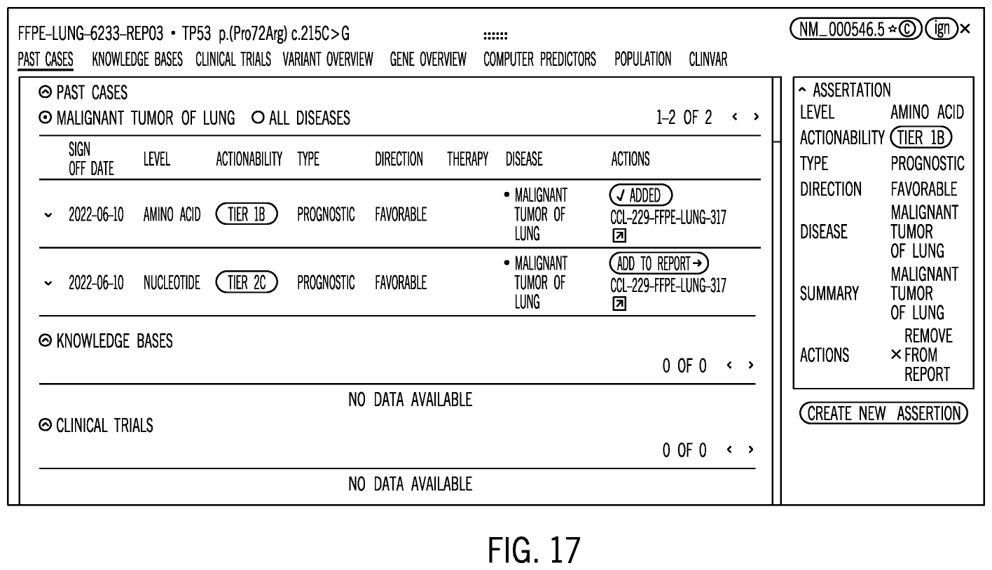

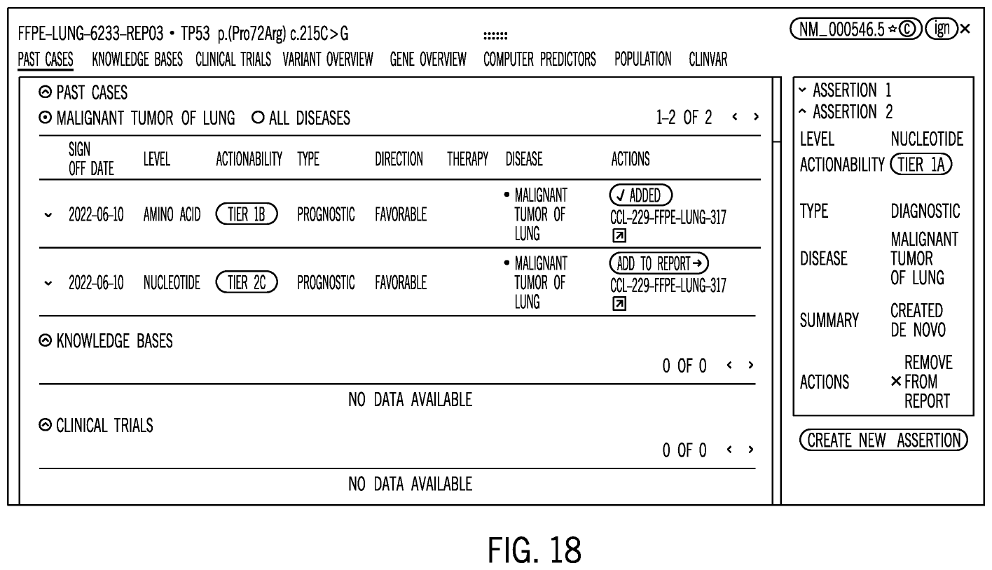

Turning to FIG. 17, a sample interface (e.g., screen) is illustrated showing the result of the user selecting to add the first past case finding to the report as an assertion. This can be seen in the Action column and, on the right-side of the screen as an “Assertion 1” where the details associated with the selected past case are added as an assertion to the present case and selected variant undergoing interpretation. Also shown in FIG. 17, an option for the user to create a new assertion is provided, allowing a user the option to create a de novo assertion if desired. This is illustrated in FIG. 18, where a second assertion (i.e., “Assertion 2”) is shown as being added and as having been created de novo by the user.

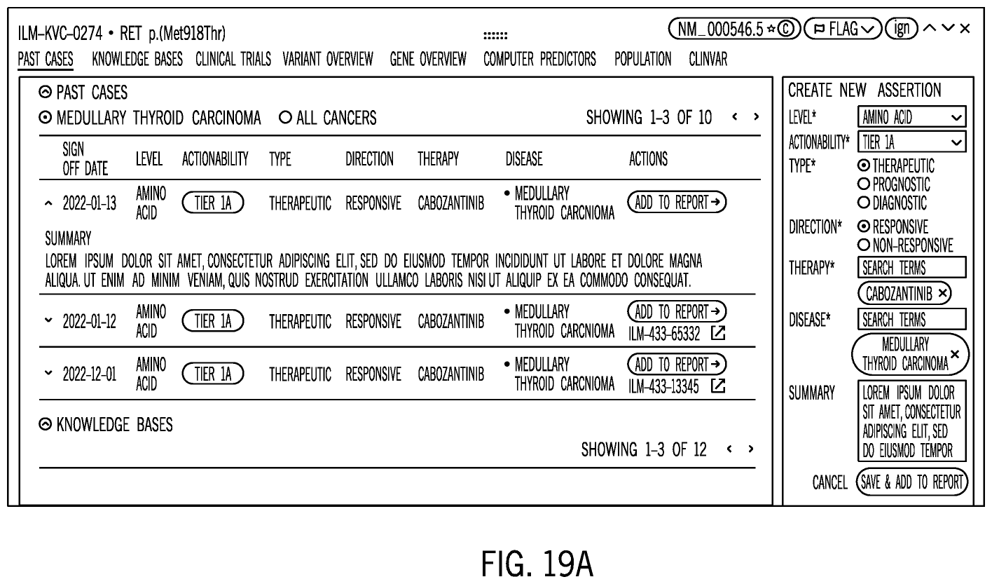

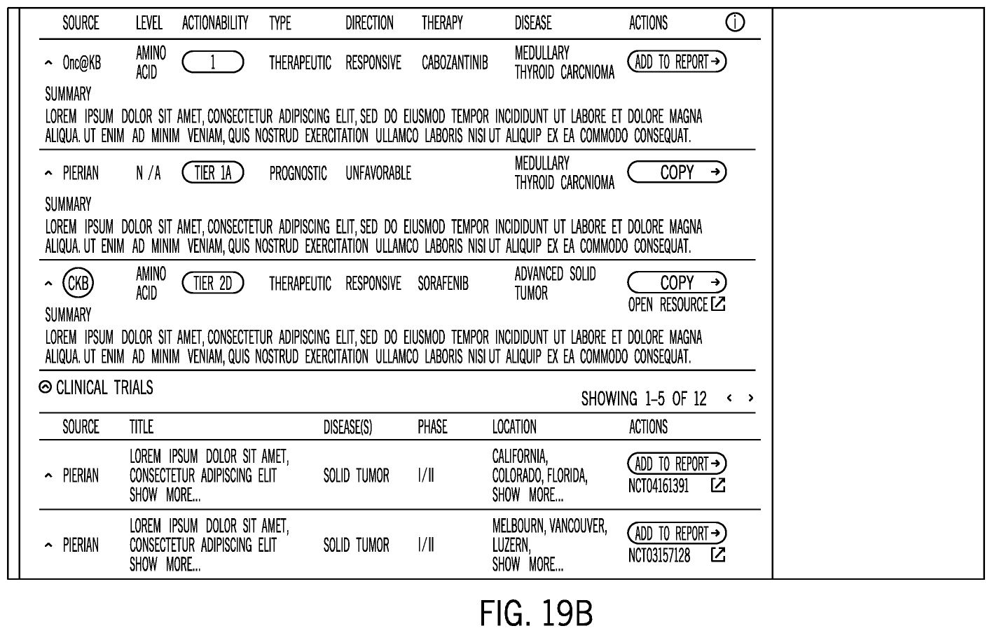

With respect to assertions and assertion creation, FIGS. 19A and 19B illustrate a further example of an assertion creation form which may be interacted with by a user to create a new assertion. In this example interface screen, the user may interact with one or more fillable (e.g., free-text) or selectable (e.g., drop down menus, radio buttons, toggles, and so forth) fields for specifying details of the new assertion. By way of example, fields for the level of the assertion (e.g., amino acid, nucleotide, and so forth), the actionability of the assertion (e.g., a category or characterization corresponding to a patient or subject work flow), the type of assertion (e.g., therapeutic, prognostic, diagnostic), the direction of the assertion (e.g., responsive or non-responsive), the therapy associated with the assertion, the disease, and a summary for the assertion.

In addition, FIGS. 19A and 19B expand on the preceding examples showing additional detailed views of external knowledge base findings related to the selected variant that may be added, in modified or unmodified form, as assertions to the case with respect to the variant. As shown in this example, each external knowledge base finding is illustrated with a source indication so as to allow a user to readily identify the source of the finding. Further, as may be seen in this example, three sources of external knowledge base findings are shown. In practice, each external knowledge base source may have a different layout in terms of fields and ontology. In the depicted example, however, findings are mapped to a normalized (e.g., common or shared) layout so as to facilitate review by the user. With the preceding descriptions of FIGS. 16-19B in mind and the depicted assertion creation form shown in FIGS. 19A and 19B, it may be appreciated that a user may create assertions in a straightforward manner based on past case data, external database information, and/or as a new (i.e., de novo) matter. That is, the software platform 156 provides flexibility to the user to facilitate creation of assertions from a single interface or screen based on three different sources (i.e., past case, external databases, or new, de novo assertions). In addition, FIGS. 19A and 19B depict clinical trials relevant to the selected variant. As shown, the clinical trials, as with the knowledge base and past case findings, may be added to the report as assertions if deemed of interest or relevant by the user.

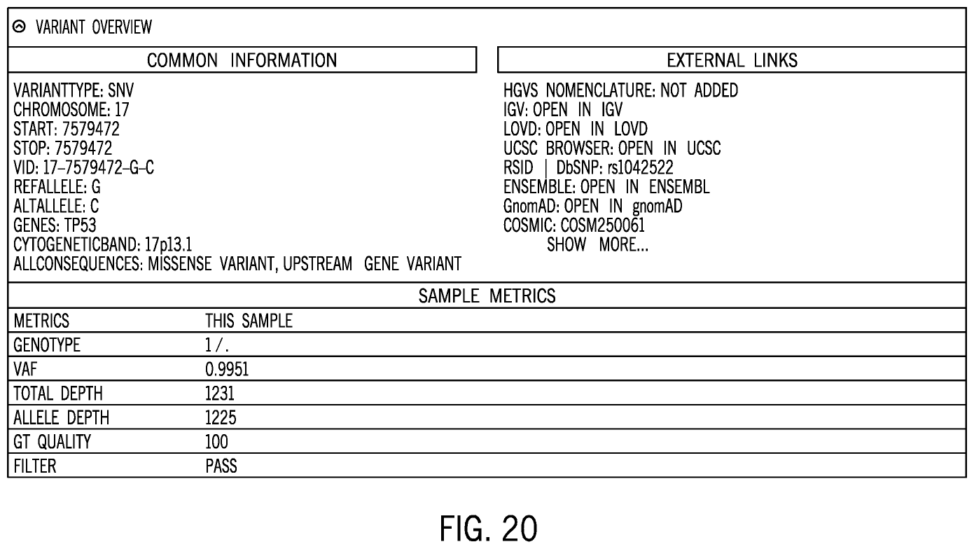

Turning to FIG. 20, a variant overview or summary screen is illustrated which can be accessed as a tab of the variant detail screen of FIGS. 16-19B. In the depicted variant overview, common information for the variant is depicted as being displayed (e.g., variant type, chromosome, start and stop positions, references allele, alternative allele, gene, cytogenetic band, consequences and so forth). In addition, external links (i.e., links to external data sources) are also provided for the variant. Sample metrics (e.g., variant allele frequency, genotype, total depth, allele depth, GT quality, and so forth) may also be included, as illustrated, as part of the variant overview information.

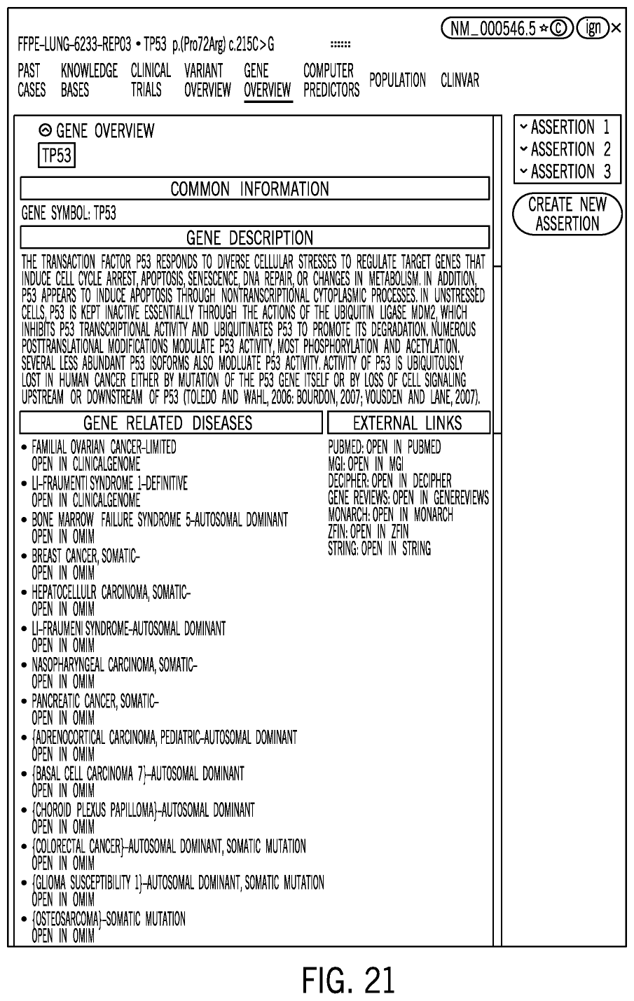

Turning to FIG. 21, a gene overview or summary screen is illustrated which can be accessed as a tab of the variant detail screen of FIGS. 16-19B and which provides information about the gene in which the selected variant is located. In the depicted gene overview, common information about the gene is depicted as being displayed. In addition, a gene description, information about diseases related or linked to the gene, and external links (i.e., links to external data sources) are also provided for the gene.

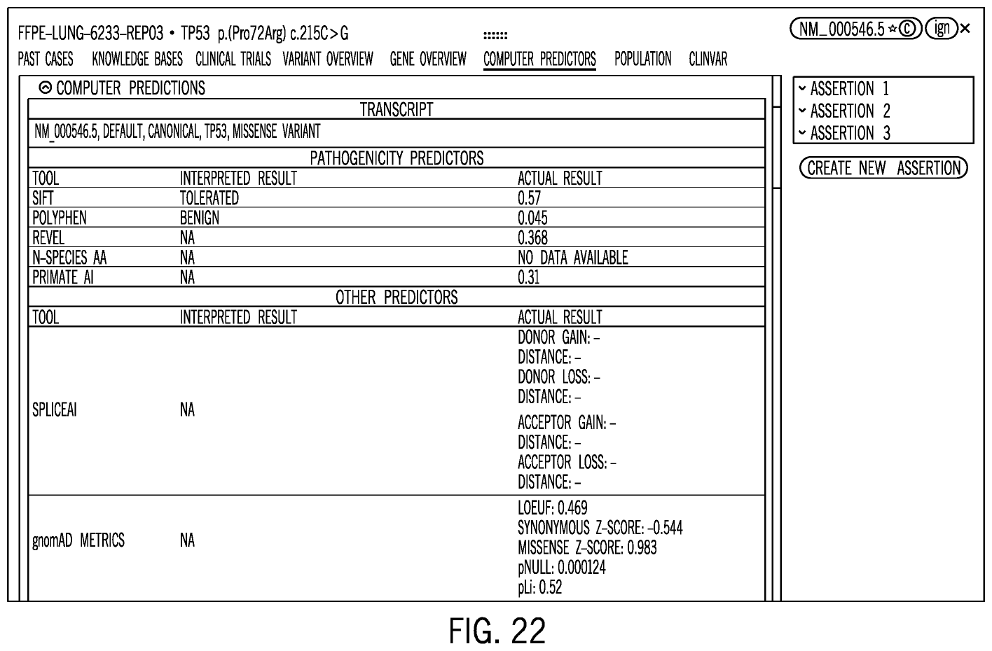

Turning to FIG. 22, computer predictors for the selected variant are illustrated which can be accessed as a tab of the variant detail screen of FIGS. 16-19B and which provide prediction information about the selected variant. In the depicted example, a transcript can be accessed related to the computer predictor of the selected variant. In addition, pathogenicity predictors (based on respective pathogenicity prediction tools and showing interpreted and actual results) and other predictors (based on corresponding tools and showing interpreted and actual results) are also provided for the selected variant.

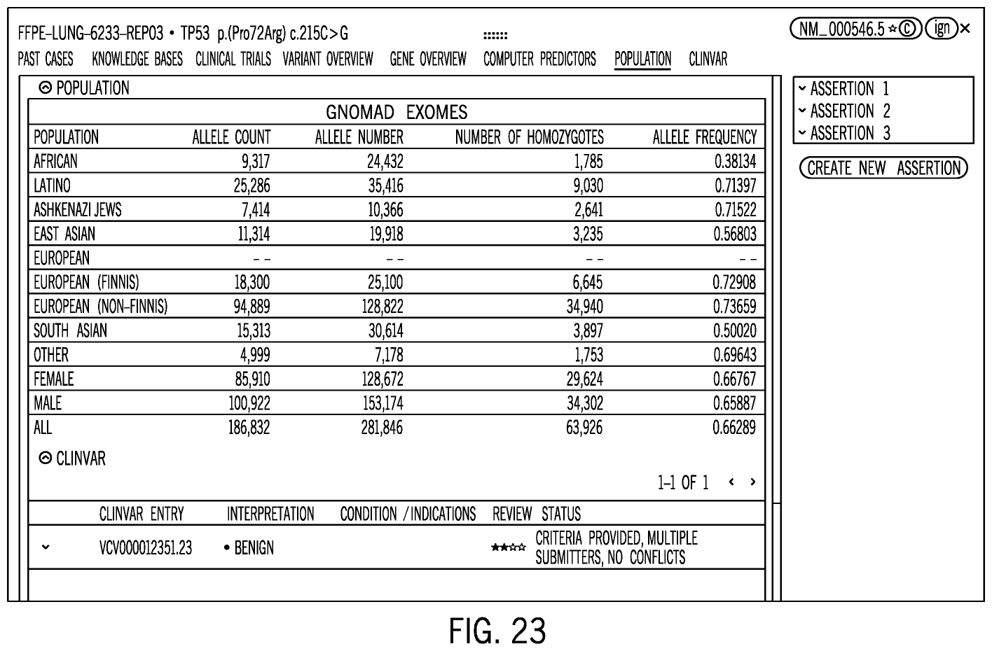

Turning to FIG. 23, population data (e.g., gnomad exomes) for the selected variant are illustrated which can be accessed as a tab of the variant detail screen of FIGS. 16-19B. In the depicted example, the population data may provide information regarding the selected variant based on different populations. Such information may include, but is not limited to, allele count, allele number, number of homozygotes, and allele frequency. In addition, FIG. 23 also depicts ClinVar data for the selected variant, which is also selectable as a tab of the variant detail screen. Such ClinVar data may include, but is not limited to, an interpretation, conditions/indications, and a review status listed for relevant ClinVar entries.

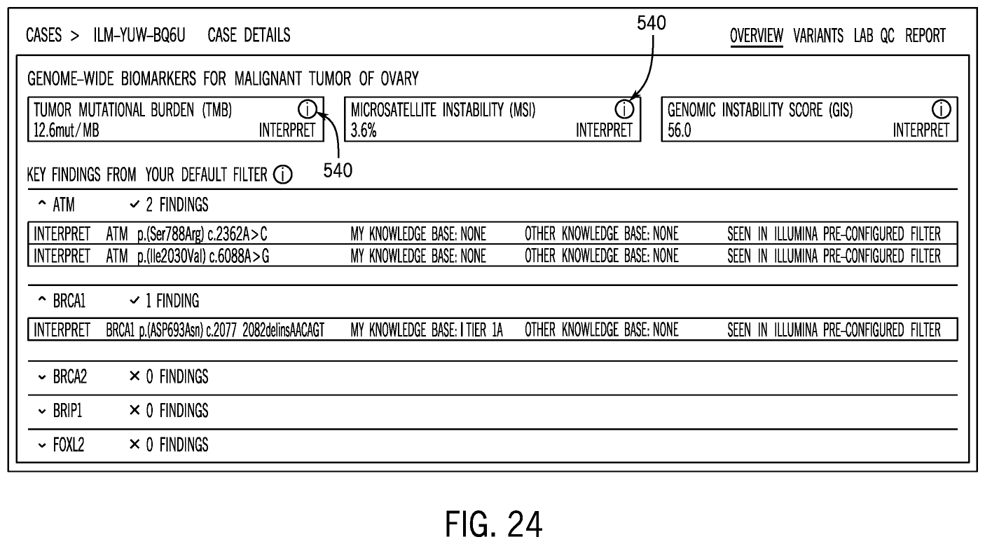

Turning to FIG. 24, a case detail overview for a selected case is depicted which can be accessed via selection of a respective case. In the depicted case detail overview, a set of genome-wide biomarkers may be displayed as part of the overview for the respective case. By way of example, three genome-wide biomarkers are illustrated as being displayed: Tumor Mutational Burden (TMB), Microsatellite Instability (MSI), and Genomic Instability Score (GIS). As displayed, each biomarker has a corresponding quantitative value or score for the selected case or aspect of the selected case that may be of use to a reviewer or may be considered for inclusion in the final report. In the depicted example, each biomarker has a corresponding selectable “Interpret” feature 540, which when selected by a user provides or displays additional information regarding the selected biomarker in the context of the selected case.

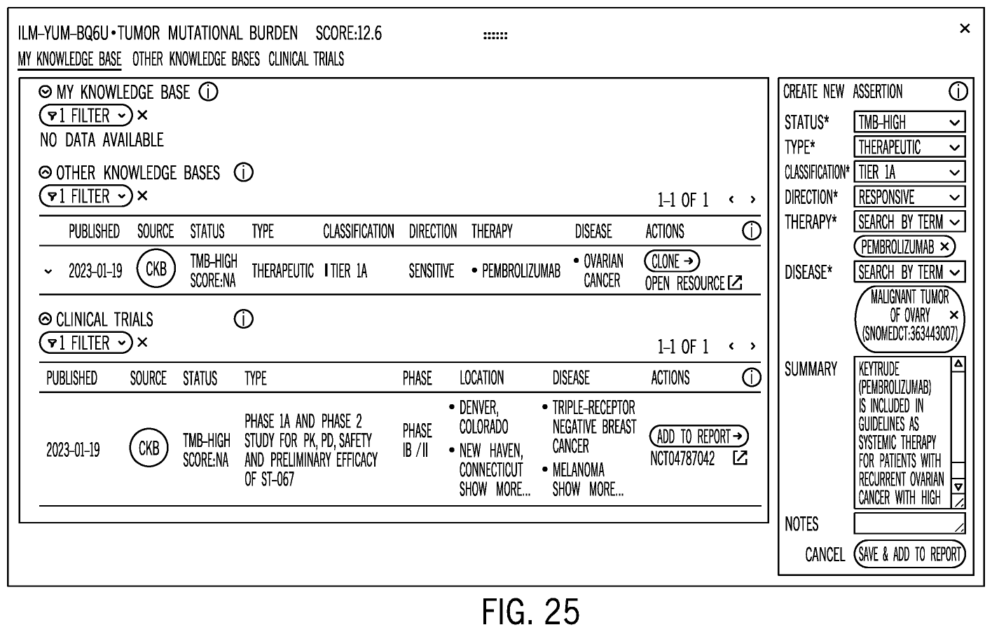

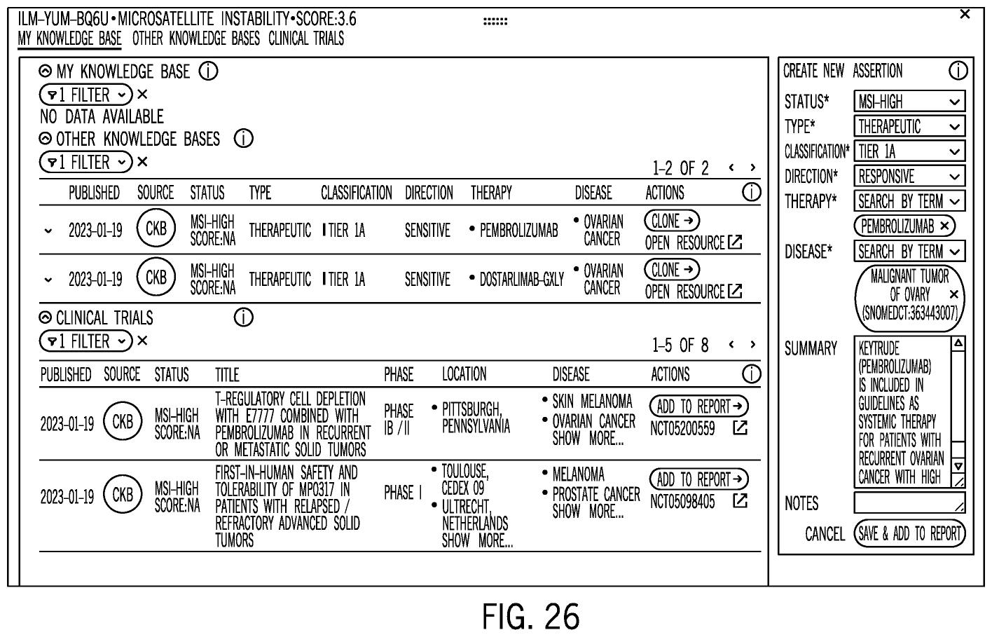

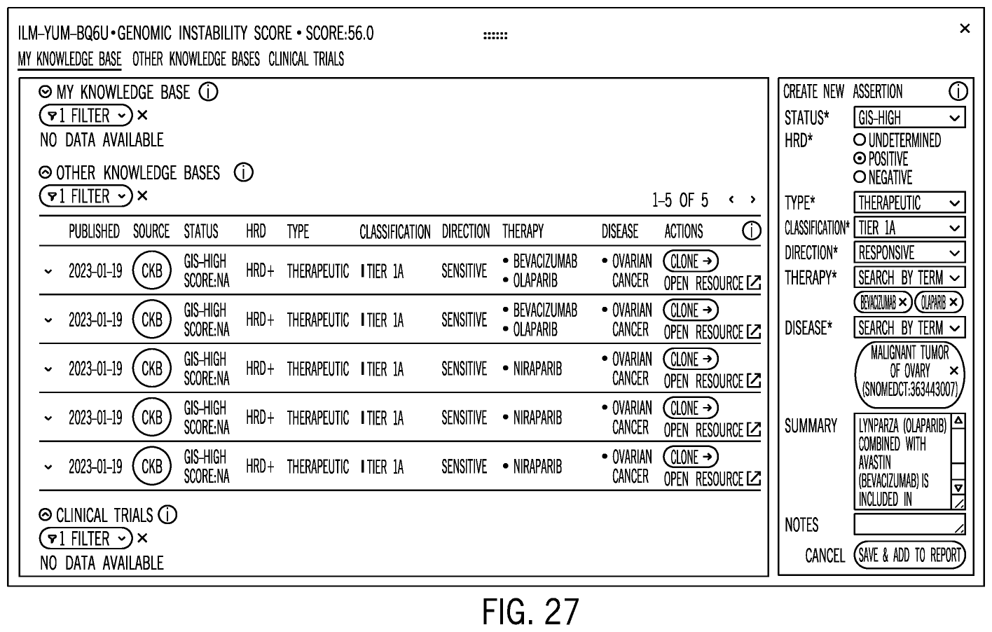

By way of example, and turning to FIG. 25, upon selection of the TMB biomarker Interpret option, a view or screen may be displayed illustrating factors or contributors of the TMB score for the respective case. In certain embodiments and as depicted in the present example, for the respective TMB score the contributors to the respective TMB score may be categorized or ordered based on source (e.g., a personalized knowledge base, external knowledgebases, clinical trials, and so forth). In the depicted example, various fields (e.g., columns) are displayed, including a publication date, a source identifier, a status (e.g., a qualitative indication of relative score, such as high, medium, or low), a type (e.g., therapeutic, diagnostic, and so forth), a classification, a direction, an indicated therapy, an associated disease, a trial title, a trial phase, a trial location, and selectable actions (e.g., actions to add an assertion to a report, actions to clone the entry, and so forth). In addition, for the biomarker, here the TMB biomarker, an option may be provided to the user to create a new assertion, as shown on the right-hand side of FIG. 25. In such an assertion creation context, entry fields may be provided for a status, a type, a classification (e.g., tier), a direction, an indicated therapy, an associated disease, a summary field, and a notes field. Upon creation of an assertion the assertion may be saved and added to the draft or final report. Turning to FIGS. 26 and 27, similar biomarker interpretation views or screens are displayed for other biomarkers, respectively MSI (FIG. 26) and GIS (FIG. 27).

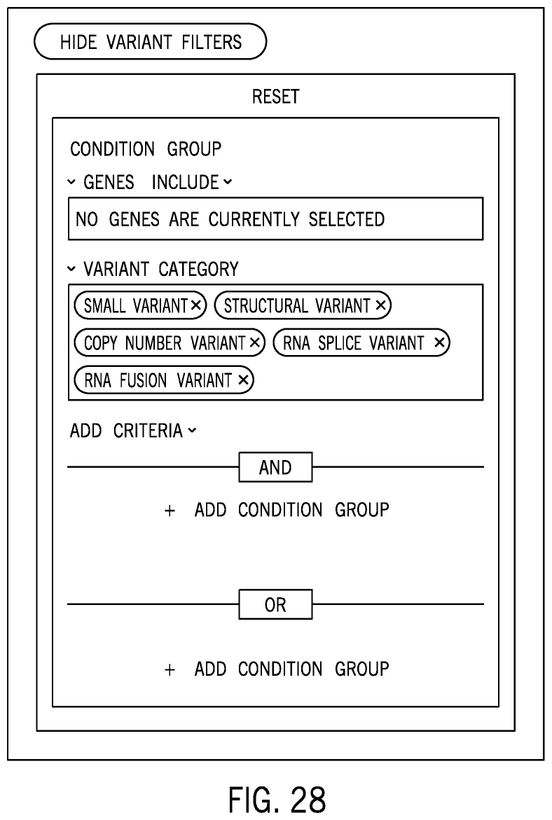

Turning to FIG. 28, an example of a filter screen is provided by which a user may filter one or both of genes or variants present in a sample based on one or more criteria specified by the user. In the depicted example, the user may utilize or specify Boolean operators as part of the filter operation so as to create multi-level and/or hierarchical filter conditions.

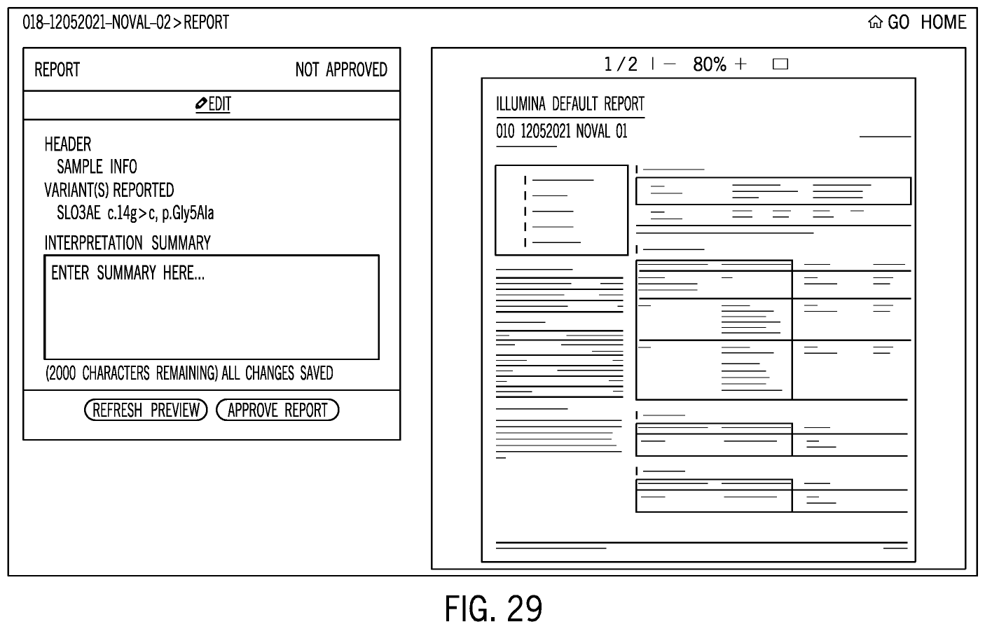

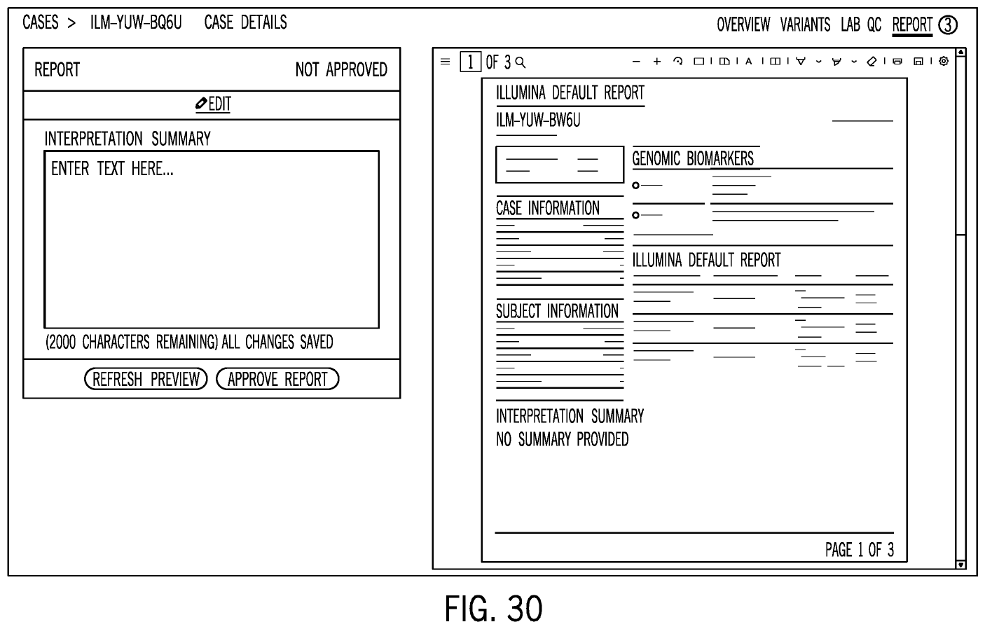

Turning to FIG. 29, an example of a draft report as may be displayed by the software platform 156 is illustrated. Such a draft report may be displayed “pre-approval” to allow a user to review, edit, or otherwise alter or revisit the report prior to signing off on the report. As discussed herein, the layout and appearance of the report may be customized by the user of the software platform 156. Such a report may be generated using assertions selected or created by the user during the interpretation stage of the tertiary analysis performed on the sample. Similarly, and turning to FIG. 30, a further example of a draft report is illustrated. In the example depicted in FIG. 30 genomic biomarker data is included (e.g., TMB, MSI, and GIS scores) along with assertions related to the biomarkers.

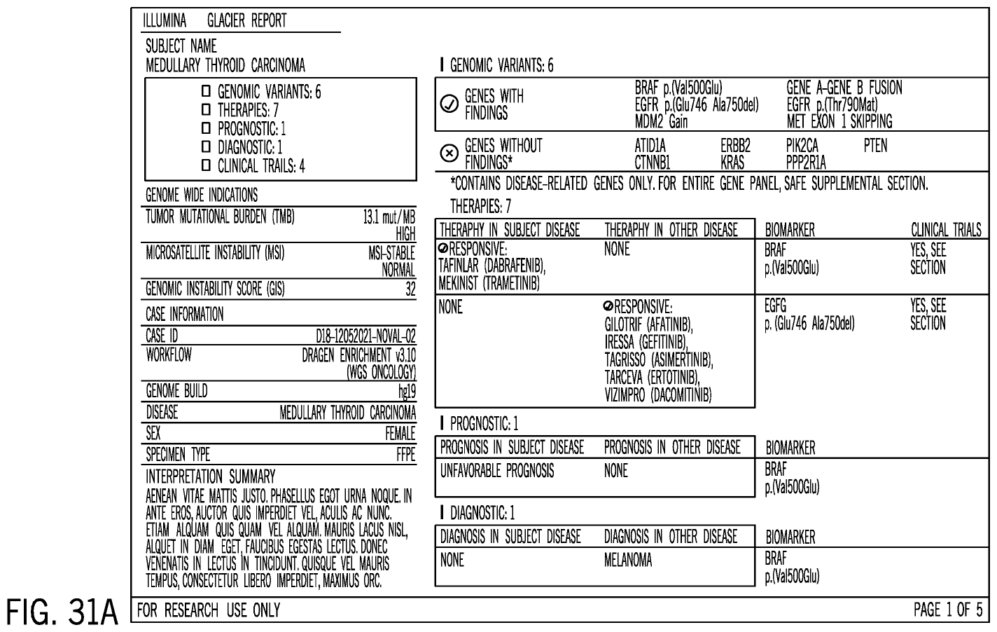

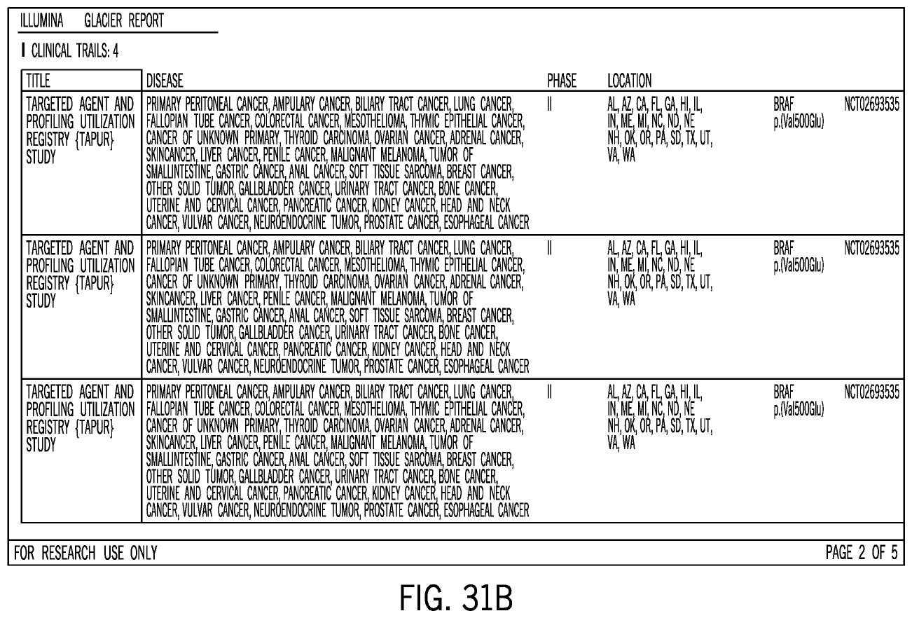

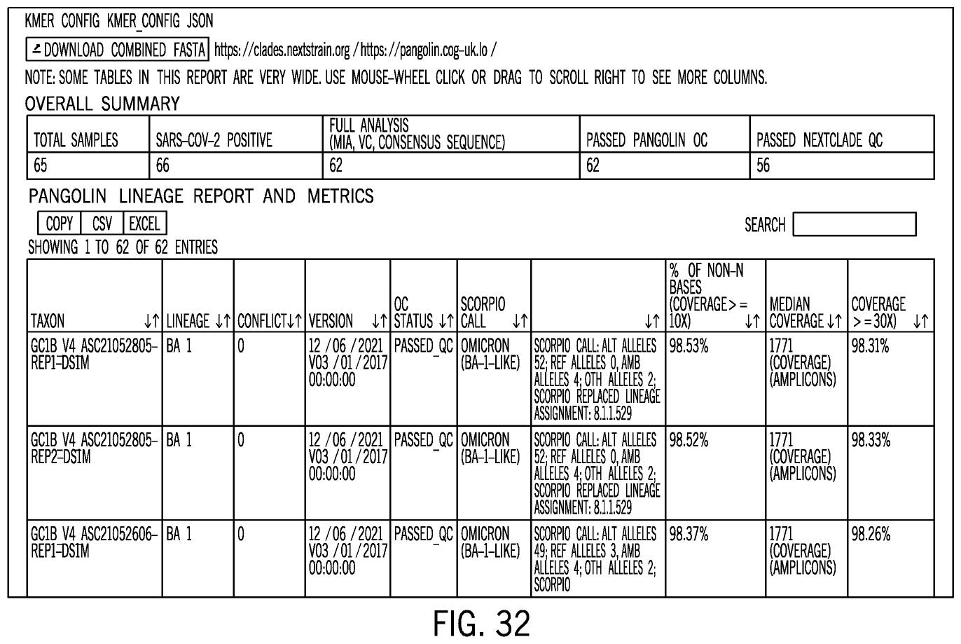

Turning to FIGS. 31A and 31B, an example of an approved report is illustrated. As depicted, such a report may include a section pertaining to genetic variants of interest, possible or recommended therapies, prognostic assertions, diagnostic assertions, and clinical trial data. In addition, genome wide indications, case information, and an interpretation summary may be included as part of the report. Turning to FIG. 32, an alternative report for a different application, here viral lineage, is illustrated. As may be appreciated, different applications (i.e., use cases) may correspondingly have different report formats or contents suited to the goals of the application.

As discussed herein, the described techniques provide for a software platform (e.g., a software application) that provides tools for users to store, arrange, and visualize genetic data, such as may be derived from a nucleic acid sequencing device. In addition, such a software platform may include one or more tools that allow a user to annotate genetic data with information available from external and/or internal genetic databases and to create custom reports based on such information. In practice, the software platform may be generic with respect to the sequencing device generating the sequence data, one or more upstream analytic packages, such as may perform variant identification or calling, and one or more external or internal data stores (e.g., knowledge bases or databases) used to access information about the sequence and/or variants identified therein.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. One or more computer readable media comprising machine-executable routines, wherein the machine-executable routines, when executed, cause acts to be performed comprising:
receiving as a first input a nucleic acid sequence dataset, wherein the nucleic acid sequence data set is an output of one or both of a primary analysis or a secondary analysis;
displaying a selectable listing of one or more variants identified in the nucleic acid sequence dataset;
receiving a selection of a variant of interest from the selectable listing of the one or more variants;
accessing one or more data stores comprising variant data associated with the selected variant of interest;
displaying one or more variant findings accessed from the one or more data stores;
receiving a selection of one or more of the variant findings;
creating an assertion for the variant of interest for each selection of the one or more variant findings; and
generating a customized report based on the assertions.

2. The one or more computer readable media of claim 1, wherein the nucleic acid sequence dataset comprises a FASTA file or a VCF.

3. The one or more computer readable media of claim 1, wherein the one or more machine-executable routines, when executed, cause further acts to be performed comprising:
displaying a genomics viewer tool configured to display visual information associated with the selected variant of interest.

4. The one or more computer readable media of claim 1, wherein the one or more machine-executable routines, when executed, cause further acts to be performed comprising:
receiving an indication of a sequence analysis application based on which the nucleic acid sequence dataset will be analyzed.

5. The one or more computer readable media of claim 4, wherein the sequence analysis application comprises one of oncology testing, environmental surveillance, anti-microbial resistance (AMR) studies, infectious disease studies, public health and microbial surveillance, genetic disorder studies, or genetic disease testing.

6. The one or more computer readable media of claim 1, wherein the one or more data stores are accessed using a cloud platform or on premise.

7. The one or more computer readable media of claim 1, wherein the one or more data stores are selected to be accessed based on a sequence analysis application.

8. The one or more computer readable media of claim 1, wherein the machine-executable routines, when executed, cause further acts to be performed comprising:
creating additional assertions based on user inputs, wherein the additional assertions are created de novo.

9. The one or more computer readable media of claim 1, wherein the one or more data stores comprise external or third-party data stores.

10. The one or more computer readable media of claim 1, wherein the one or more data stores comprise an internal data store comprising past history case data for a user or an organization with which the user is affiliated.

11. The one or more computer readable media of claim 10, wherein the internal data store comprises a personalized knowledge base.

12. The one or more computer readable media of claim 11, wherein the machine-executable routines, when executed, cause further acts to be performed comprising:
providing a template for entry of past history case data for the user or the organization with which the user is affiliated;
receiving as an input a populated template; and
processing the populated template to generate or update the personalized knowledge base.

13. The one or more computer readable media of claim 1, wherein the nucleic acid sequence dataset comprises or is derived from sequence data of human origin.

14. The one or more computer readable media of claim 1, wherein the nucleic acid sequence dataset comprises or is derived from sequence data of non-human origin.

15. The one or more computer readable media of claim 1, wherein the one or more variant findings accessed from the one or more data stores are displayed in a normalized layout.

16. The one or more computer readable media of claim 1, wherein the nucleic acid sequence dataset is automatically uploaded from a location that is continuously or periodically monitored and that is specified by a user input.

17. The one or more computer readable media of claim 1, wherein the one or more machine-executable routines, when executed, cause further acts to be performed comprising:
displaying one or more genome-wide biomarkers derived for the nucleic acid sequence dataset or variants present in the nucleic acid sequence dataset, wherein each genome-wide biomarker is displayed with an associated score and wherein one or more of the genome-wide biomarkers is selectable to create assertions used to generate the customized report.

18. One or more computer readable media comprising machine-executable routines, wherein the machine-executable routines, when executed, cause acts to be performed comprising:
accessing or receiving a data file comprising genetic data for a subject, wherein the genetic data comprises one or both of a primary analysis or a secondary analysis of the subject's genetic composition; and
generating a variant details summary for display or printout, wherein the variant details summary integrates and concurrently shows data comprising:
external variant detail data acquired from one or more data stores external to a machine executing the machine-readable instructions; and
local variant detail data comprising past case data of a user of the machine or an organization to which the user belongs.

19. The one or more computer readable media of claim 18, wherein the external variant detail data and the local variant detail data are displayed or printed having a shared field layout.

20. The one or more computer readable media of claim 18, wherein the machine-executable routines, when executed, cause further acts to be performed comprising:
generating a customizable report based on the genetic data, wherein the report comprises one or more assertions generated automatically or by the user, wherein each assertion relates one or more variant or disease observations derived from the genetic data to data derived from the one or more data stores external to the machine or from past case data.

21. The one or more computer readable media of claim 20, wherein the one or more assertions comprise one or more of a therapeutic assertion, a prognostic assertion, or a diagnostic assertion.

22. The one or more computer readable media of claim 18, wherein the machine-executable routines, when executed, cause further acts to be performed comprising:
displaying an actionability criteria for one or more variant or disease characterizations derived from the genetic data accessed, wherein the actionability criteria are customizable by the user or the organization to which the user belongs.

23. The one or more computer readable media of claim 22, wherein the actionability criteria, when applied, specify a workflow for a given subject having a respective genetic variant or disease.

24. The one or more computer readable media of claim 18, wherein the local variant detail data is accessed from a personalized knowledge base.

25. One or more computer readable media comprising machine-executable routines, wherein the machine-executable routines, when executed, cause acts to be performed comprising:
receiving as a first input a nucleic acid sequence dataset, wherein the nucleic acid sequence data set is an output of one or both of a primary analysis or a secondary analysis;
displaying a selectable listing of one or more variants identified in the nucleic acid sequence dataset;
receiving a selection of a variant of interest from the selectable listing of the one or more variants;
accessing two or more external data stores comprising variant data associated with the selected variant of interest;
displaying one or more variant findings accessed from the two or more external data stores;
receiving a selection of one or more of the variant findings;
creating an assertion for the variant of interest for each selection of the one or more variant findings; and
generating a customized report based on the assertions.

26. The one or more computer readable media of claim 25, wherein the one or more machine-executable routines, when executed, cause further acts to be performed comprising:
receiving an indication of a sequence analysis application based on which the nucleic acid sequence dataset will be analyzed.

27. The one or more computer readable media of claim 26, wherein the sequence analysis application comprises one of oncology testing, environmental surveillance, anti-microbial resistance (AMR) studies, infectious disease studies, public health and microbial surveillance, genetic disorder studies, or genetic disease testing.

28. The one or more computer readable media of claim 25, wherein the two or more external data stores are accessed using a cloud platform.

29. The one or more computer readable media of claim 25, wherein the two or more data stores are selected to be accessed based on a sequence analysis application.

30. The one or more computer readable media of claim 25, wherein the machine-executable routines, when executed, cause further acts to be performed comprising:
creating additional assertions based on user inputs as de novo assertions or based on selected past case data.

31. The one or more computer readable media of claim 25, wherein the one or more variant findings accessed from the one or more data stores are displayed in a normalized layout.

32. One or more computer readable media comprising machine-executable routines, wherein the machine-executable routines, when executed, cause acts to be performed comprising:
receiving as a first input a nucleic acid sequence dataset, wherein the nucleic acid sequence data set is an output of one or both of a primary analysis or a secondary analysis;
displaying a selectable listing of one or more variants identified in the nucleic acid sequence dataset;
receiving a selection of a variant of interest from the selectable listing of the one or more variants;
accessing past case data comprising variant data associated with the selected variant of interest;
displaying one or more variant findings accessed from the past case data;
receiving a selection of one or more of the variant findings;
creating an assertion for the variant of interest for each selection of the one or more variant findings; and
generating a customized report based on the assertions.

33. The one or more computer readable media of claim 32, wherein the one or more machine-executable routines, when executed, cause further acts to be performed comprising:
receiving an indication of a sequence analysis application based on which the nucleic acid sequence dataset will be analyzed.

34. The one or more computer readable media of claim 33, wherein the sequence analysis application comprises one of oncology testing, environmental surveillance, anti-microbial resistance (AMR) studies, infectious disease studies, public health and microbial surveillance, genetic disorder studies, or genetic disease testing.

35. The one or more computer readable media of claim 32, wherein the machine-executable routines, when executed, cause further acts to be performed comprising:
creating additional assertions based on user inputs as de novo assertions or based on data accessed from one or more external data stores.

36. One or more computer readable media comprising machine-executable routines, wherein the machine-executable routines, when executed, cause acts to be performed comprising:
accessing or receiving a data file comprising genetic data for a subject, wherein the genetic data comprises one or both of a primary analysis or a secondary analysis of the subject's genetic composition; and
displaying a variant details summary interface, wherein the variant details summary interface integrates and concurrently shows data comprising:
external variant detail data acquired from one or more data stores external to a machine executing the machine-readable instructions; and
local variant detail data comprising past case data of a user of the machine or an organization to which the user belongs; and
providing on or via the variant details summary interface selectable options for creating one or more assertions based on the external variant detail data, the local variant detail data, or a de novo assertion entry.
